(19) United States
(12) Patent Application Publication
(10) Pub. No.: US 2015/0002490 A1
(43) Pub. Date: Jan. 1, 2015

(54) METHOD OF MOVING THE DISPLAYS OF AN ULTRASOUND DIAGNOSTIC DEVICE AND ULTRASOUND DIAGNOSTIC DEVICE

(71) Applicant: SAMSUNG ELECTRONICS CO., LTD., Suwon-si (KR)

(72) Inventors: Jeong-ho HAN, Seoul (KR); Eun-ho YANG, Seoul (KR)

(73) Assignee: SAMSUNG ELECTRONICS CO., LTD., Suwon-si (KR)

(21) Appl. No.: 14/314,313

(22) Filed: Jun. 25, 2014

(30) Foreign Application Priority Data
Jun. 28, 2013 (KR) 10-2013-0075944

(51) Int. Cl. A61B 8/00

(52) U.S. Cl. CPC A61B 8/462
USPC 345/204; 345/31

(57) ABSTRACT

A method of moving a display of an ultrasound diagnostic device includes obtaining an image of an operator capturing an ultrasound image of an object by using the ultrasound diagnostic device, determining a position and a viewing direction of the operator based on the image of the operator, and changing a position of the display connected to the ultrasound diagnostic device from a first position to a second position according to the position and the viewing direction of the operator.

### CROSS-REFERENCE TO RELATED PATENT APPLICATION

This application claims the benefit of Korean Patent Application No. 10-2013-0075944, filed on Jun. 28, 2013, in the Korean Intellectual Property Office, the disclosure of which is incorporated herein in its entirety by reference.

### BACKGROUND

1. Field

Exemplary embodiments relate to a method of moving a display of an ultrasound diagnostic device and to an ultrasound diagnostic device, and more particularly, to a method of moving a display of an ultrasound diagnostic device based on an operator's viewing direction, and to an ultrasound diagnostic device.

2. Description of the Related Art

An ultrasound diagnostic device is used to observe the internal structure of an organic body. The ultrasound diagnostic device is non-invasive testing equipment that shows structural detailed items, internal tissues, and flow of a liquid in a human body.

The ultrasound diagnostic device irradiates an ultrasound signal generated from a transducer of a probe to an object and receives information of a response signal reflected from the object, thereby obtaining an image of the interior of the object. In particular, the ultrasound diagnostic device is used for a medical purpose such as observation of the interior of an object, detection of foreign materials, and measurement of an injury, etc.

The ultrasound diagnostic device exhibits high stability. Further, the ultrasound diagnostic methods provide more safety than other imaging methods, such as X-ray and CT imaging methods, due to the fact that ultrasound diagnostic methods do not involve exposure to hazardous radiation such as X-rays. Also, since real-time image display is possible, the ultrasound diagnostic device is widely used with other imaging diagnostic devices.

### SUMMARY

Exemplary embodiments provide a method of moving a display of an ultrasound diagnostic device, and an ultrasound diagnostic device, which enables an operator who uses the ultrasound diagnostic device to operate a probe and simultaneously easily check a display.

According to a first exemplary embodiment, a method of moving a display of an ultrasound diagnostic device includes obtaining an image of an operator capturing an ultrasound image of an object by using the ultrasound diagnostic device, determining a position and a viewing direction of the operator based on the image of the operator, and changing a position of the display connected to the ultrasound diagnostic device from a first position to a second position according to the position and the viewing direction of the operator.

The changing of the position of the display may include determining a visible range of the operator based on the position and the viewing direction of the operator, and changing the position of the display from the first position to the second position so that a screen of the display is located in the determined visible range of the operator.

The determining of the viewing direction of the operator may include determining a posture of the operator based on the image of the operator, and determining the viewing direction of the operator by using the determined posture of the operator.

The determining of the posture of the operator may include determining the posture of the operator based on at least one of a shape and a positional relationship of a head part and a body part of the operator included in the image of the operator.

The image of the operator may include a support table supporting the object, and the determining of the viewing direction of the operator may include determining the viewing direction of the operator in consideration of a positional relationship between the operator and the support table included in the image of the operator.

The changing of the position of the display may include determining the second position in consideration of a positional relationship between the ultrasound diagnostic device and the operator.

The changing of the position of the display may include determining a position located in the visible range of the operator in a space within which the display is movable, as the second position.

The obtaining of the image of the operator may include obtaining a depth image of the operator, and the determining of the position and the viewing direction of the operator includes determining the position and the viewing direction of the operator based on the depth image of the operator.

The obtaining of the depth image may include obtaining a depth image of the operator by using a depth camera.

The obtaining of the depth image may include obtaining images of the operator by using at least two color cameras, and obtaining a depth image of the operator by applying stereo matching to the images of the operator obtained by using the at least two color cameras.

The changing of the position of the display includes determining a movement path from the first position to the second position based on information about a position of at least one of a person and an object included in the image of the operator.

The determining of the movement path may include determining the movement path from the first position to the second position to guide the display not to collide against at least one of the person and the object included in the image of the operator.

The changing of the position of the display may include changing a movement path of the display when it is determined that an obstacle exists on the movement path of the display while the position of the display is changed from the first position to the second position.

The method may further include obtaining information about a portion of the object, obtaining an image of the object, determining a position of a portion corresponding to the portion in the image of the object, and changing a position of the display from a third position to the first position according to the determined position of the portion.

The obtaining of the image of the object may include obtaining a depth image of the object.

The determining of the position and the viewing direction of the operator may include determining a person who is the closest to a position of a probe of the ultrasound diagnostic device when a plurality of persons are included in the image of the operator.

The determining of the person who is the closest to the position of the probe of the ultrasound diagnostic device as the operator may include determining the position of the probe by using infrared or short-rang communication, and determining a person who is the closest to the position of the probe based on the position of the probe and the image of the operator, as the operator.

The display may include a first display and a second display, and the method further including adjusting an angle between the first display and the second display.

The adjusting of the angle between the first display and the second display may include adjusting the angle between the first display and the second display so that a screen of the first display and a screen of the second display are located at the viewing direction of the operator.

The adjusting of the angle between the first display and the second display may include adjusting the angle between the first display and the second display so that a screen of the first display is located at the viewing direction of the operator and the screen of the second display is located at a viewing direction of the object.

The method may further include adjusting at least one of a brightness value, a contrast value, and a tilting angle of the display located at the second position according to a preset value.

The method may further include detecting gesture of the operator with respect to the display located at the second position, and performing a function of the ultrasound diagnostic device corresponding to the gesture of the operator.

The detecting of the gesture of the operator may include detecting gesture of the operator by using the image of the operator.

The detecting of the gesture of the operator may include performing a function corresponding to a position indicated by a hand of the operator among functions displayed on the display when it is determined that the hand of the operator and the display are located within a preset distance for a preset time, based on the image of the operator.

The detecting of the gesture of the operator may include detecting gesture of the operator by using a gesture detection camera attached to the display.

The detecting of the gesture of the operator may include detecting gesture of an eye of the operator by using an eye tracking sensor attached to the display, and performing a function of the ultrasound diagnostic device corresponding to the detected gesture of the eye of the operator.

The method may further include receiving a display position change input by the operator.

The display position change input may include at least one of an input through a foot switch connected to the ultrasound diagnostic device, an input through operator's voice, and an input through operator's gesture.

The method may further includes receiving a capturing end input by the operator, and changing the position of the display from the second position to the first position according to the capturing end input.

According to another exemplary embodiment, a method of moving a display of an ultrasound diagnostic device includes obtaining an image of an object whose ultrasound image is to be captured by using the ultrasound diagnostic device, obtaining information about a portion of the object, determining a position of a portion corresponding to the portion based on the image of the object, and changing a position of the display connected to the ultrasound diagnostic device from a first position to a second position according to the determined position of the portion.

According to another exemplary embodiment, a method of moving a display of an ultrasound diagnostic device includes obtaining an image of an operator who captures an ultrasound image of an object by using the ultrasound diagnostic device, i.e. operates the ultrasonic device to capture the ultrasonic images, determining a viewing direction of the operator from the obtained image of the operator, and adjusting an angle between a first display and a second display that are connected to the ultrasound diagnostic device, according to the viewing direction of the operator.

The adjusting of the angle between the first display and the second display may include adjusting the angle between the first display and the second display so that a screen of the first display and a screen of the second display are located at the viewing direction of the operator.

The method may further include obtaining an image of the object, and determining a viewing direction of the object based on the image of the object, wherein the adjusting of the angle between the first display and the second display includes adjusting the angle between the first display and the second display so that a screen of the first display is located at the viewing direction of the operator and the screen of the second display is located at a viewing direction of the object.

According to another exemplary embodiment, there is provided a non-transitory computer readable recording medium having recorded thereon a program for executing the above method.

According to another exemplary embodiment, an ultrasound diagnostic device includes an image obtaining unit obtaining an image of an operator capturing an ultrasound image of an object, a determination unit determining a position and a viewing direction of the operator based on the image of the operator, a display displaying at least one of information of the object and an ultrasound image of the object, and a control unit changing a position of the display from a first position to a second position according to the position and the viewing direction of the operator.

The determination unit may determine a visible range of the operator based on the position and the viewing direction of the operator, and the control unit may change the position of the display from the first position to the second position so that a screen of the display is located within the visible range of the operator.

The determination unit may determine a posture of the operator based on the image of the operator and determines the viewing direction of the operator by using the determined posture of the operator.

The determination unit may determine the posture of the operator based on at least one of a shape and a positional relationship of a head part and a body part of the operator included in the image of the operator.

The image of the operator may include a support table supporting the object, and the determination unit may determine the viewing direction of the operator in consideration of a positional relationship between the operator and the support table included in the image of the operator.

The control unit may determine the second position in consideration of a positional relationship between the ultrasound diagnostic device and the operator.

The control unit may determine a position located in the visible range of the operator in a space within which the display is movable, as the second position.

The image obtaining unit may obtain a depth image of the operator, and the determination unit may determine the position and the viewing direction of the operator based on the depth image of the operator.

The image obtaining unit may include a depth camera for obtaining a depth image of the operator.

The image obtaining unit may include at least two color cameras for obtaining images of the operator and obtain a depth image of the operator by applying stereo matching to the images of the operator obtained by using the at least two color cameras.

The control unit may determine a movement path from the first position to the second position based on information about a position of at least one of a person and an object included in the image of the operator.

The control unit may determine the movement path from the first position to the second position to guide the display not to collide against at least one of the person and the object included in the image of the operator.

The display may include a distance detection sensor that detects an obstacle located within a predetermined distance.

The control unit may change the movement path of the display when an obstacle detected by the distance detection sensor exists on the movement path of the display while the position of the display is changed from the first position to the second position.

The ultrasound diagnostic device may further include a communication unit obtaining information about a portion of the object, wherein the image obtaining unit obtains an image of the object, the determination unit may determine a position of a portion corresponding to the portion in the image of the object, and the control unit may change a position of the display from a third position to the first position according to a determined position of the portion.

The determination unit may determine a person who is the closest to a position of a probe of the ultrasound diagnostic device when a plurality of persons are included in the image of the operator.

The ultrasound diagnostic device may further include an infrared emitting unit that emits an infrared ray, in which the determination unit determines a position of an object that reflects or absorbs the infrared ray emitted by the infrared emitting unit, as a position of the probe.

The display may include a first display and a second display, and the control unit may adjust an angle between the first display and the second display.

The control unit may adjust the angle between the first display and the second display so that a screen of the first display and a screen of the second display are located at the viewing direction of the operator.

The control unit may adjust the angle between the first display and the second display so that a screen of the first display is located at the viewing direction of the operator and the screen of the second display is located at a viewing direction of the object.

The control unit may adjust at least one of a brightness value, a contrast value, and a tilting angle of the display located at the second position according to a preset value.

The ultrasound diagnostic device may further include a gesture detection unit that detects gesture of the operator with respect to the display located at the second position, in which the control unit performs a function of the ultrasound diagnostic device corresponding to the gesture of the operator.

The gesture detection unit may detect gesture of the operator by using the image of the operator.

The gesture detection unit may determine whether a hand of the operator and the display are located within a preset distance for a preset time, based on the image of the operator, and the control unit may perform a function corresponding to a position indicated by the hand of the operator among functions displayed on the display.

The gesture detection unit may include a gesture detection camera that is attached on the display and detects gesture of the operator.

The gesture detection unit may include an eye tracking sensor that is attached to the display and detects gesture of an eye of the operator, and the control unit may perform a function of the ultrasound diagnostic device corresponding to the detected gesture of the eye of the operator.

The ultrasound diagnostic device may further include a communication unit that receives a display position change input by the operator.

The display position change input may include at least one of an input through a foot switch connected to the ultrasound diagnostic device, an input through operator's voice, and an input through operator's gesture.

The ultrasound diagnostic device may further include a communication unit that receives a capturing end input by the operator, in which the control unit changes the position of the display from the second position to the first position according to the capturing end input.

According to exemplary embodiments, an ultrasound diagnostic device includes an image obtaining unit obtaining an image of an object whose ultrasound image is to be captured, a communication unit obtaining information about a portion of the object, a determination unit determining a position of a portion corresponding to the portion based on the image of the object, a display displaying at least one of information of the object and an ultrasound image of the object, and a control unit changing a position of the display from a first position to a second position according to the determined position of the portion.

According to another exemplary embodiment, an ultrasound diagnostic device includes an image obtaining unit obtaining an image of an operator who captures an ultrasound image of an object, a determination unit determining a viewing direction of the operator from the image of the operator, a first display and a second display displaying at least one of information of the object and the ultrasound image of the object, and a control unit adjusting an angle between the first display and the second display that are connected to the ultrasound diagnostic device, according to the viewing direction of the operator.

The control unit may adjust the angle between the first display and the second display so that a screen of the first display and a screen of the second display are located at the viewing direction of the operator.

The image obtaining unit may obtain an image of the object, the determination unit may determine a viewing direction of the object based on the image of the object, and the control unit may adjust the angle between the first display and the second display so that a screen of the first display is located at the viewing direction of the operator and the screen of the second display is located at a viewing direction of the object.

The image obtaining unit may obtain a depth image of the operator, and the determination unit may determine the viewing direction of the operator from the depth image of the operator.

The foregoing general description and the following detailed description are only exemplary and explanatory.

### BRIEF DESCRIPTION OF THE DRAWINGS

The above and/or other features and advantages of the exemplary embodiments will become more apparent by describing in detail exemplary embodiments thereof with reference to the attached drawings in which:

FIG. 1 illustrates an environment in which an operator operates a general ultrasound diagnostic device;

FIG. 2 illustrates the operation of an ultrasound diagnostic device according to an exemplary embodiment;

FIG. 3 is a flowchart for explaining a method of moving a display of an ultrasound diagnostic device, according to an exemplary embodiment;

FIG. 4 is a flowchart for explaining Operation S310 of FIG. 3 in detail;

FIG. 5 illustrates a method of determining an operator from a depth image including a plurality of persons;

FIG. 6 is a flowchart for explaining Operation S320 of FIG. 3 in detail;

FIG. 7A illustrates a depth image;

FIG. 7B illustrates a method of determining a viewing direction of a person included in the depth image;

FIG. 8 illustrates another method of determining a viewing direction of an operator from a depth image;

FIG. 9 illustrates another method of determining a viewing direction of an operator from a depth image;

FIG. 10 is a flowchart for explaining Operation S330 of FIG. 3 in detail;

FIG. 11 illustrates a method of determining a second position considering a positional relationship between an ultrasound diagnostic device and an operator;

FIG. 12 illustrates a method of determining a movement path between a first position and the second position from a depth image;

FIG. 13 is a flowchart for explaining a method of moving a display of an ultrasound diagnostic device, according to another exemplary embodiment;

FIG. 14 illustrates a method of moving a display from a third position to the first position;

FIG. 15 is a flowchart for explaining a method of moving a display of an ultrasound diagnostic device, according to another exemplary embodiment;

FIG. 16 is a flowchart for explaining a method of moving a display of an ultrasound diagnostic device, according to another exemplary embodiment;

FIG. 17 illustrates a method of adjusting an angle between a first display and a second display;

FIG. 18 illustrates another method of adjusting an angle between the first display and the second display;

FIG. 19A illustrates a method of detecting gesture of an operator from a depth image;

FIG. 19B illustrates a method of performing a function of an ultrasound diagnostic device displayed on a display according to operator's gesture;

FIG. 20 is a block diagram illustrating the structure of an ultrasound diagnostic device according to an exemplary embodiment;

FIG. 21 is a block diagram illustrating the structure of an ultrasound diagnostic device according to another exemplary embodiment; and

FIG. 22 is a block diagram illustrating the structure of a wireless probe that may be connected to an ultrasound diagnostic device, according to an exemplary embodiment.

### DETAILED DESCRIPTION

The following detailed description is provided to gain a comprehensive understanding of the methods, apparatuses and systems described herein. Various changes, modifications, and equivalents of the systems, apparatuses and methods described herein will suggest themselves to those of ordinary skill in the art. Descriptions of well-known functions and structures are omitted to enhance clarity and conciseness.

The attached drawings for illustrating exemplary embodiments are referred to in order to gain a sufficient understanding, the merits thereof, and the objectives accomplished by the implementation of exemplary embodiments. Hereinafter, exemplary embodiments will be described in greater detail with reference to the attached drawings. Like reference numerals in the drawings denote like elements.

Terms used in the present specification will be briefly described. The terminology used herein is for the purpose of describing particular embodiments only and is not intended to be limiting of the present disclosure.

General terms that are currently widely used are selected as the terms used for the present specification in consideration of functionality in the present specification and availability. However, the terms may vary according to intention of engineers working in the present technical field, precedents, advent of new technology, etc. Also, in a specific case, the applicant particularly selects terms and in such a case the meaning of a term is defined in detail in the detailed description section. Accordingly, the terms used for the present specification should be defined based on the meaning of the term and the overall context of the present specification, not simply by a nominal meaning of the term.

When a part may “include” a certain constituent element, unless specified otherwise, it may not be construed to exclude another constituent element but may be construed to further include other constituent elements. The terms such as “portion”, “unit”, “module”, and “block” stated in the specification may signify a unit to process at least one function or operation and the unit may be embodied by hardware such as FPGA or ASIC, software, or a combination of hardware and software. However, the term “portion” in the present specification is not limited by a specific combination of hardware and software. The “portion” may be configured in a storage medium that may be addressed or to be able to reproduce one or more processors. Accordingly, as an example, the “portion” includes constituent elements such as software constituent elements, object-oriented software constituent elements, class constituent elements, and task constitute elements, processes, functions, attributes, procedures, subroutines, segments of a program code, drivers, firmware, micro-codes, circuits, data, database, data structures, tables, arrays, and variables. Functions provided in the constituent elements and the “portions” may be combined into a smaller number of constituent elements or the “portions” or classified into additional constituent elements and the “portions”.

As used herein, the term “and/or” includes any and all combinations of one or more of the associated listed items. Expressions such as “at least one of,” when preceding a list of elements, modify the entire list of elements and do not modify the individual elements of the list. Although some features may be described with respect to individual exemplary embodiments, aspects need not be limited thereto such that features from one or more exemplary embodiments may be combinable with other features from one or more exemplary embodiments.

In the present specification, an “image” may signify multi-dimensional data formed of discrete image elements (for example, pixels in a two-dimensional image and voxels in a three-dimensional image). For example, the image may include a medical image of an object obtained by X-ray, CT, MRI, ultrasound, and other medical imaging systems.

Also, in the present specification, an “object” may include the body of a human or an animal, or a part thereof. For example, the object may include internal organs such as liver, heart, womb, brain, breasts, abdomen, etc. or blood vessels. Also, the “object” may include phantom. The phantom may signify a substance having a density of a living thing and a volume closely approximate to the effective atom number and may include spherical phantom having a similar feature to a human body.

Also, in the present specification, a “operator” may be a medical expert, for example, a medical doctor, a nurse, a clinical technologist, a medical imaging expert, etc. and a technologist who fixes medical equipment, but the exemplary embodiments are not limited thereto.

Also, in the present specification, an “ultrasound image” signifies an image about an object obtained by using an ultrasonic wave.

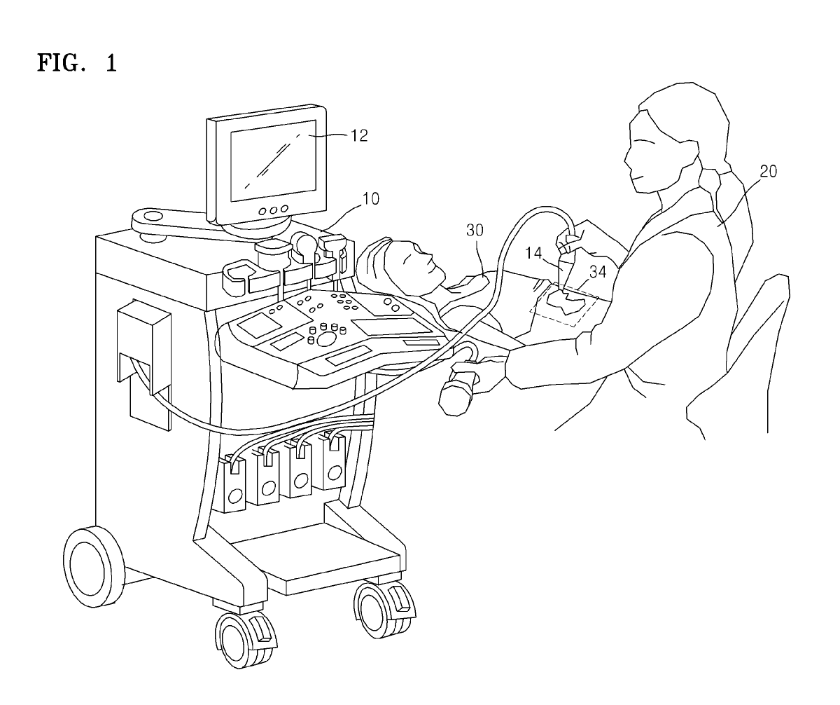

FIG. 1 illustrates an environment in which an operator 20 operates a general ultrasound diagnostic device 10. Referring to FIG. 1, the general ultrasound diagnostic device 10 includes a display 12 displaying an ultrasound image of an object 30 and a probe 14 transmitting an ultrasound signal to the object 30 and receiving a response signal reflected from the object 30. The operator 20 locates the probe 14 to a portion 34 of the object 30 to obtain an image of the interior of the object 30 and observes an ultrasound image displayed on the display 12.

In the general ultrasound diagnostic device 10, since the display 12 is fixedly attached on the general ultrasound diagnostic device 10, if the portion 34 of the object 30 and the display 12 of the general ultrasound diagnostic device 10 are located in different directions, the operator 20 needs to continuously change a viewing direction between the portion 34 of the object 30 and a screen of the display 12. This may be very inconvenient for the operator 20 to diagnose the object 30.

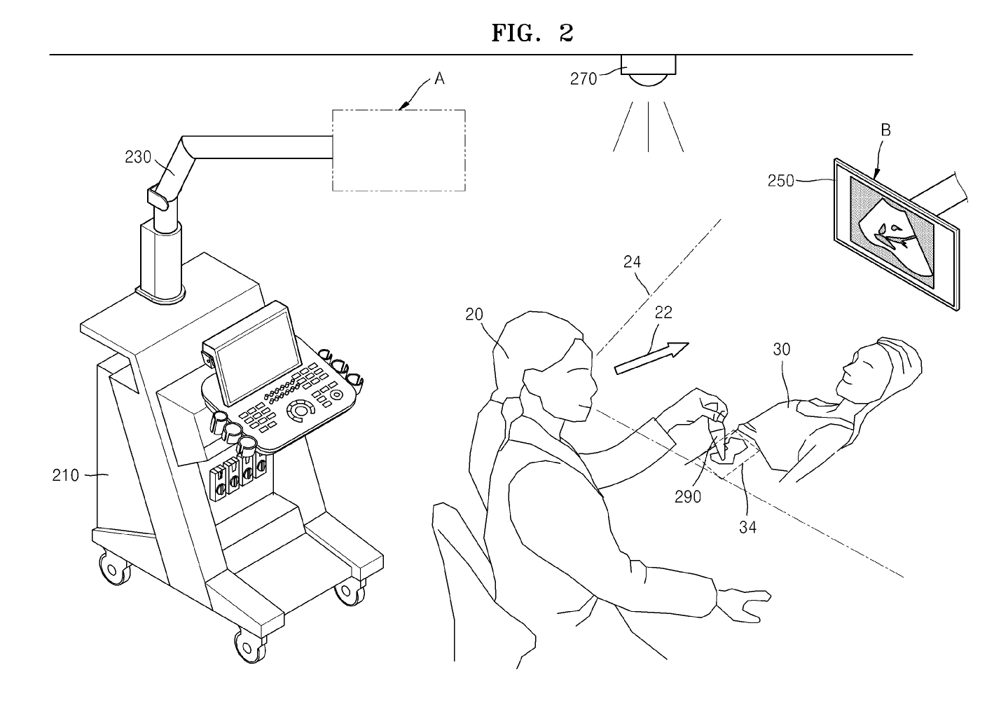

FIG. 2 illustrates the operation of an ultrasound diagnostic device 210 according to an exemplary embodiment. The ultrasound diagnostic device 210 according to the present embodiment determines a viewing direction 22 of the operator 20 based on an image of the operator 20 obtained by a black and white camera, a depth camera, or a color camera 270. Next, the ultrasound diagnostic device 210 may change the position of a display 250 according to the determined viewing direction 22 from a first position A to a second position B.

The ultrasound diagnostic device 210 and the display 250 may be connected by a multi-joint arm 230. The multi-joint arm 230 may have a degree of freedom of at least 6 axes and may move the display 250 in a direction and to a position that the operator 20 desires. The structure of the multi-joint arm 230 in FIG. 2 is an example and the ultrasound diagnostic device 210 may include an arm having various structures that may move the position of the display 250.

Referring to FIG. 2, the display 250 of the ultrasound diagnostic device 210 is moved from the first position A to the second position B and thus the portion 34 of the object 30 and a screen of the display 250 both may be located within a visible range 24 of the operator 20. Accordingly, the operator 20 may operate a probe 290 and simultaneously easily check the screen of the display 250.

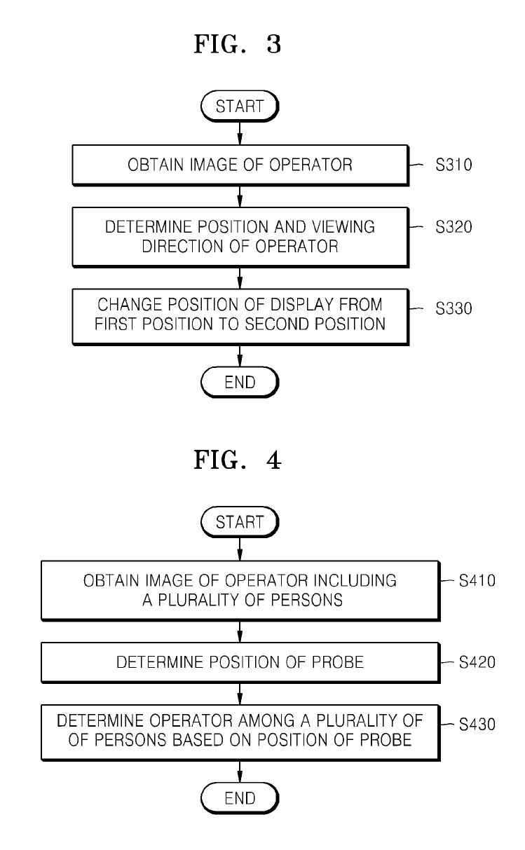

FIG. 3 is a flowchart for explaining a method of moving a display of an ultrasound diagnostic device, according to an exemplary embodiment. Referring to FIG. 3, in Operation S310, the ultrasound diagnostic device 210 obtains an image of an operator. The ultrasound diagnostic device 210 may continuously obtain images of an operator at a predetermined time interval.

The image of an operator is obtained by using a color camera, a black and white camera, or a depth camera. Also, the image of an operator may include a depth image of an operator. The depth image signifies an image including information about a distance between a camera capturing a depth image and an object captured by the camera. The images may be obtained by a plurality of cameras disposed at various positions in the ultrasound diagnostic room. For example, cameras may be disposed on the walls, ceiling, or stands (the stands may be used to adjust the positions of the camera). Further, cameras may be attached to specific parts of the ultrasound diagnostic apparatus. For example, cameras may be attached or incorporated to a side of the displays.

The ultrasound diagnostic device 210 according to the present embodiment may receive an image of an operator from an external device or server or obtain an image of an operator by using a color camera, a black and white camera, or a depth camera.

Also, the ultrasound diagnostic device 210 may obtain a depth image of an operator by using a depth camera or obtain a depth image by applying stereo matching to images of an operator obtained from at least two color cameras.

A method of obtaining a depth image including information about a distance of an object by applying stereo matching to the image of an operator obtained by using at least two color cameras is well known to one of ordinary skill in the art and thus a detailed description thereof will be omitted in the present specification.

In Operation S320, the ultrasound diagnostic device 210 determines a position and a viewing direction of an operator from the image of an operator. In other words, the ultrasound diagnostic device 210 determines the position where the operator is located in a predetermined space including the operator and a viewing direction or a direction in which the operator looks. A method of determining a viewing direction of an operator is described below in detail with reference to FIGS. 6 to 9. A viewing direction may refer to a direction in which the operator looks, a viewing field, or a field of view.

In Operation S330, the ultrasound diagnostic device 210 changes the position of the display 250 from a first position to a second position according to the position and viewing direction of an operator. In detail, the ultrasound diagnostic device 210 may determine a position to which the display 250 moves considering the position and viewing direction of an operator and move the position of the display 250 to the determined position. The ultrasound diagnostic device 210 may change the position of the display 250 from the first position to the second position so that the screen of the display 250 may be located within a visible range of an operator. The display may be sequentially moved through various positions. The movement of the display between two positions, such as from a first position to a second position, may be performed on various paths.

The ultrasound diagnostic device 210 may determine the second position considering a positional relationship between the operator and the ultrasound diagnostic device 210 or determine a movement path to prevent collision between the display 250 and other objects, which will be described below with reference to FIGS. 10 to 12.

When an operator takes a posture to easily observe the portion 34 of the object 30 at a certain position and the display 250 of the ultrasound diagnostic device 210 moves to the position of the operator and along the viewing direction of the operator, the portion 34 of the object 30 and the screen of the display 250 may be located altogether within the visible range of the operator. Accordingly, the operator may operate the probe 290 and simultaneously easily check the screen of the display 250.

The ultrasound diagnostic device 210 may adjust at least one of a brightness value, a contrast value, and a tilting angle of the display 250 located at the second position according to a preset value. When at least one of the brightness value, the contrast value, and the tilting angle of the display 250 is preset by an operator, at least one of the brightness value, the contrast value, and the tilting angle of the display 250 located at the second position is adjusted according to the preset value and thus operator convenience may be improved.

FIG. 4 is a flowchart for explaining Operation S310 of FIG. 3 in detail. In detail, FIG. 4 is a flowchart for explaining a method of determining an operator when a plurality of persons are included in an image of an operator.

In Operation S410, the ultrasound diagnostic device 210 obtains an image of an operator. The image may include a plurality of persons. As described above, the image of an operator may be received from an external device or server or may be obtained from a color camera, a black and white camera, or a depth camera.

In Operation S420, the ultrasound diagnostic device 210 determined the position of the probe 290 from the image of an operator. The ultrasound diagnostic device 210 may determine the position of the probe 290 in a variety of methods. For example, the ultrasound diagnostic device 210 may determine the position of the probe 290 by identifying the probe 290 from the image of an operator. When the probe 290 may not be identified from the image of an operator, the ultrasound diagnostic device 210 may determine the position of the probe 290 in a predetermined space by using an infrared ray that is emitted into the predetermined space and detecting a reflected or absorbed infrared ray. An infrared reflection portion for reflecting an infrared ray or an infrared absorption portion for absorbing an infrared ray may be attached on the probe 290. Also, the ultrasound diagnostic device 210 may determined the position of the probe 290 by using short range communication such as RFID, Bluetooth, etc. Alternatively, a particular color or shape is marked on the probe 290 and then the color or shape of the probe 290 is identified by using a color camera, thereby determining the position of the probe 290. However, the above methods for determining the position of the probe and for identifying the probe may be applied to other objects in the room where the ultrasound diagnostic device is located. The determined positions and identities of the probe and the other objects may be used such as to ensure that the displays do not collide with the objects when moving from one position to the next.

In Operation S430, the ultrasound diagnostic device 210 determines as an operator a person who is the closest to the position of the probe 290 among the persons included in the image of an operator. Since an operator using the ultrasound diagnostic device 210 may possesses the probe 290, the ultrasound diagnostic device 210 determines as the operator a person who is the closest to the position of the probe 290 among the persons included in the image of an operator. When the ultrasound diagnostic device 210 determines the position of the probe 290 by using an infrared ray, short range communication, etc., the position of the probe 290 in the predetermined space where the probe 290 is located is mapped with the image of an operator and a person who is the closest to the position of the probe 290 may be determined to be the operator.

However, the methods of identifying an operator among the persons in the image are not limited to the above described methods. For example, similar to the method of identifying the probe 290, an operator may wear a particular mark or tag that the ultrasound diagnostic device 210 may recognize via an image recognition software. The operator may be identified, among the persons included in the images, by identifying which person in the images wears the particular mark or tag. The mark or tag may be worn as a separate effect or piece on operator's clothes, body parts, eyeglasses, headset or the like. The mark or tag may be painted (using for example a particular shape, color or fluorescence) on operator's clothes, body parts, eyeglasses, headset or the like. Similarly, other persons and images in the image may have or wear identifying marks and tags. For instance, an assistant of the operator may wear another tag or mark. This way the ultrasound diagnostic device 210 may identify various persons and objects in the images. Another method for identifying an operator or for facilitating the identification of the operator among the persons included in the images may include identifying which person in the images performs a certain gesture. For instance, at the start of the diagnostic process the operator may perform a specific hand gesture (e.g. snap his or her fingers) that an image recognition software may recognize in the images recorded by the camera.

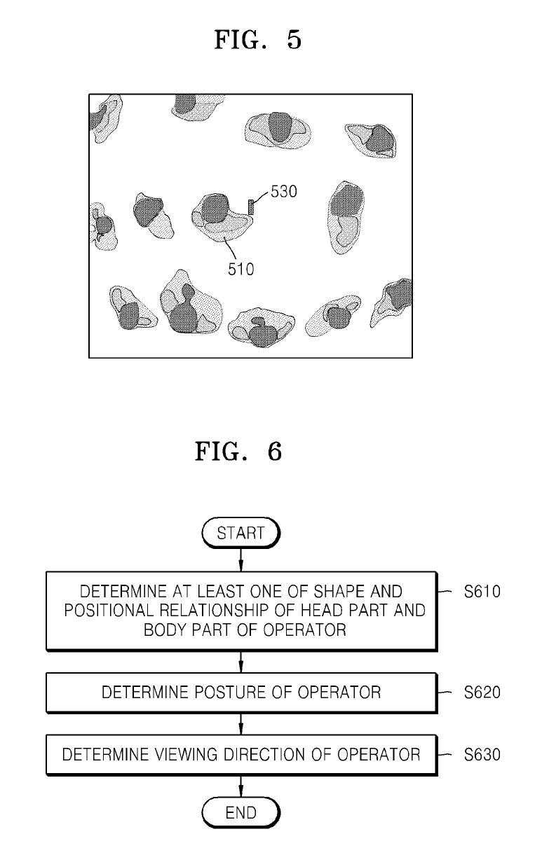

FIG. 5 illustrates a method of determining an operator from a depth image including a plurality of persons. FIG. 5 illustrates a depth image of an operator included in a plurality of persons.

The ultrasound diagnostic device 210 may identify a probe 530 in an image of an operator including a plurality of persons and determine that a person 510 who is the closest to the position of the identified probe 530 as an operator.

FIG. 6 is a flowchart for explaining Operation S320 of FIG. 3 in detail. FIG. 6 illustrates a sequence in a method of determining a viewing direction of an operator.

In Operation S610, the ultrasound diagnostic device 210 determines at least one of a shape and a positional relationship of a head part and a body part from the image of an operator. In other words, the ultrasound diagnostic device 210 may identify a head part and a body part of an operator from the image of an operator and determine at least one of the shapes of the head part and the body part that are identified and a positional relationship between the head part and the body part.

In Operation S620, the ultrasound diagnostic device 210 determines the posture of an operator based on at least one of the shapes and the positional relationship of the head part and the body part of an operator. In other words, the ultrasound diagnostic device 210 may determine whether the operator is sitting, standing, or facing a certain direction considering at least one of the shapes and the positional relationship of the head part and the body part of an operator. A method of determining the posture of an operator by using at least one of the shapes and the positional relationship of the head part and the body part of the operator is a well-known technology to one of ordinary skill in the art and thus a detailed description thereof will be omitted herein.

In Operation S630, the ultrasound diagnostic device 210 determines the viewing direction of the operator based on the posture of the operator.

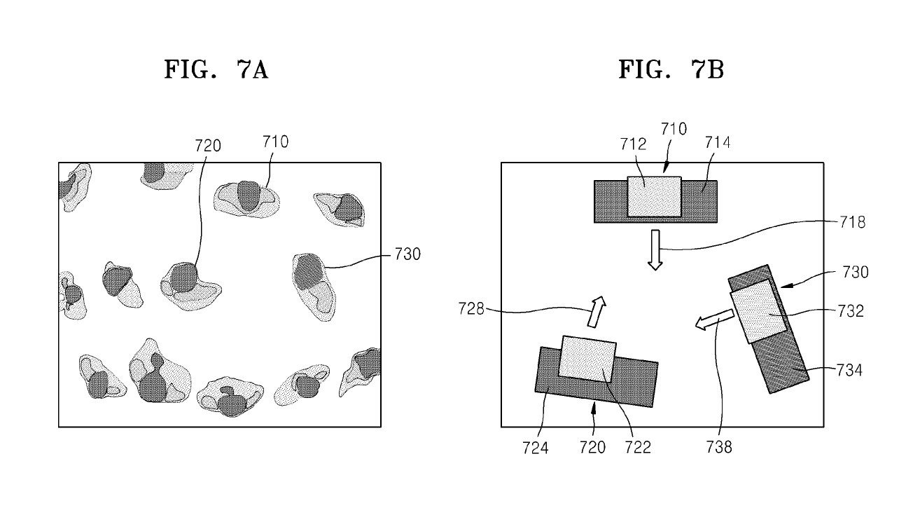

FIG. 7A illustrates a depth image and FIG. 7B illustrates a method of determining a viewing direction of a person included in the depth image.

Referring to FIG. 7A, the ultrasound diagnostic device 210 may identify head parts and body parts of persons included in a depth image by applying an image processing technique such as pattern matching, omega detection, add a boost, etc., to the depth image.

FIG. 7B schematically illustrates head parts 712, 722, and 732 and body parts 714, 724, and 734 of a first person 710, a second person 720, and a third person 730 included in the depth image of FIG. 7A. The ultrasound diagnostic device 210 may determine viewing directions 718, 728, and 738 of the first person 710, the second person 720, and the third person 730 by using at least one of the shapes and relative positional relationship of the head parts 712, 722, and 732 and the body parts 714, 724, and 734 of the first person 710, the second person 720, and the third person 730 of FIG. 7B. Referring to 7B, it can be seen that the viewing direction 718 of the first person 710, the viewing direction 728 of the second person 720, and the viewing direction 738 of the third person 730 are in the directions of six o'clock, one o'clock, and eight o'clock, respectively.

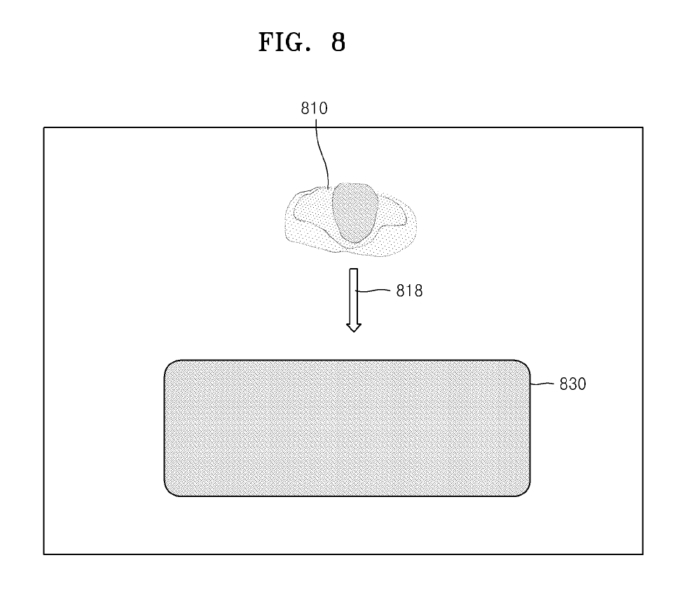

FIG. 8 illustrates another method of determining a viewing direction 818 of an operator 810 from a depth image. The depth image of the operator 810 may include a support table 830 supporting an object (not shown). Since the operator 810 takes a posture in a direction toward the object to diagnose the object, the viewing direction 818 of the operator 810 may be determined further considering a relative position between the operator 810 and the support table 830. Referring to FIG. 8, since the support table 830 is located in the direction of six o'clock of the operator 810 on the drawing sheet, the viewing direction 818 of the operator 810 may be determined to be in the direction of six o'clock.

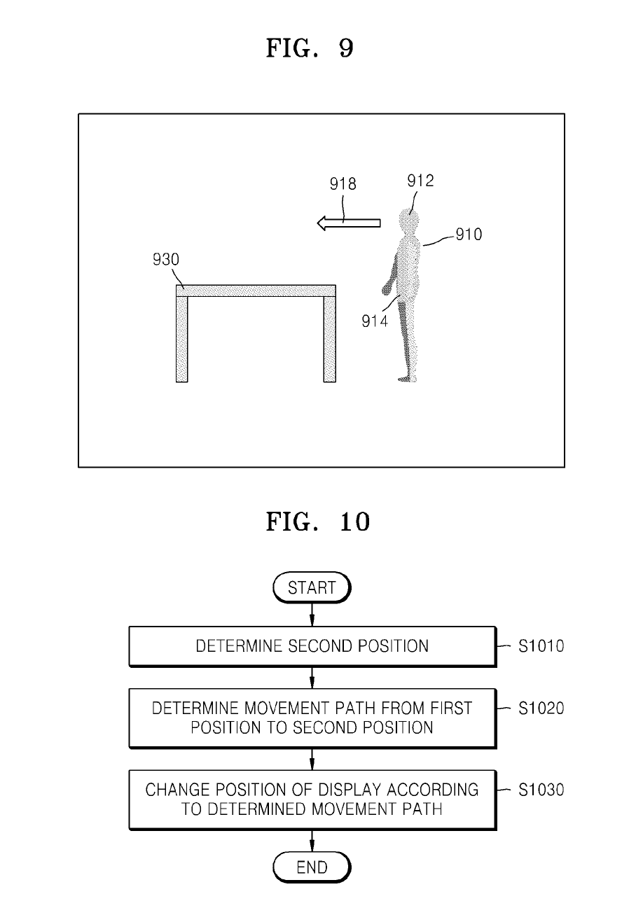

FIG. 9 illustrates another method of determining a viewing direction 918 of an operator 910 from a depth image. While FIGS. 7 and 8 illustrate a depth image by capturing an operator from the top side, that is, from the ceiling, FIG. 9 illustrates a depth image captured from a lateral direction of the operator 910. A depth camera or a color camera capturing a depth image of an operator may be connected to a main body of the ultrasound diagnostic device 210 or located at a variety of positions within a range that is obvious to one of ordinary skill in the art.

For a depth image captured in the lateral direction, the viewing direction 918 of the operator 910 may be determined based on at least one of a shape and a relative positional relationship of a head part 912 and a body part 914 of the operator 910. Also, the ultrasound diagnostic device 210 may determined the viewing direction 918 of the operator 910 further considering a relative positional relationship between the operator 910 and a support table 930. Referring to FIG. 9, since the support table 930 is located in the direction of nine o'clock of the operator 910 on the drawing sheet, the viewing direction 918 of the operator 910 may be determined to be in the direction of nine o'clock. After determining the viewing direction of an operator the ultrasound diagnostic device 210 may determine whether the operator looks at the display or whether the operator looks at other objects such as the probe, the investigated person or object, or in other directions. The position of the display may be adjusted according to whether the operator looks towards the display or towards other objects.

In FIGS. 6-9, the viewing direction of an operator is described to be determined considering the shape and positional relationship of the head part and the body part of the operator. However, such a description is a mere example and the viewing direction of an operator may be determined by using various pieces of information such as the shape of a lower body of an operator, the position of a hand of an operator, the position of a foot of an operator, etc.

Further, a viewing direction of an operator may be found by using other techniques such as described in the following. In an exemplary embodiment, an eye tracking sensor may be used to track a direction indicated by the eyeballs of the operator, this way a viewing direction may be determined. The eye tracking sensor may be disposed in the room or on the operator's body (e.g. attached on a headset or eyeglasses).

In another exemplary embodiment, similar to a method of identifying the probe 290, the operator may wear or have one or more marks or tags attached to operator's clothes, body parts, eyeglasses, headset or the like. Determining the position of the marks and tags may provide a viewing direction. For example, the operator may wear a tag on a headset or eyeglasses; the tag may have a particular orientation (e.g. may consist of an arrow, attached to the headset, pointing towards a viewing direction); a viewing direction may be found by determining the tag's position and tag's orientation using a pattern recognition software or the like. In another exemplary embodiment, the operator may have one or more small paint patches painted on one or more parts of the body (e.g. forehead, nose tip, fingers etc.); the paint may have a specific color or fluorescence that the ultrasound system may recognize; the disposition and configuration of the various paint patches on operator's bods may provide a viewing direction of the operator. The patches of paint may be used to identify various body parts of the operator.

In another exemplary embodiment, similar to the method of identifying the probe 290, the ultrasound diagnostic device 210 may determine operator's position and viewing direction by using an infrared ray that is emitted into a predetermined space and detecting a reflected or absorbed infrared ray. An infrared reflection portion for reflecting an infrared ray or an infrared absorption portion for absorbing an infrared ray may be attached on the operator. Also, the ultrasound diagnostic device 210 may determine operator's position or viewing direction by using short range communication such as RFID, Bluetooth, etc.

The methods and procedures described in this application may be combined with each other in various ways such as to obtain various methods for determining operator's viewing position and the positions of the other objects in the room. For example, the methods described in FIG. 7A may be used in conjunction with the infrared detection sensor and the imaging of the marks and tags attached to the operator. This way, various combinations of methods, procedures, and devices may be used function of the desired parameters of the ultrasound diagnostic device such as cost of the ultrasound system, performance, ease of use, room configuration, economic feasibility etc. Further, the above methods and devices may be combined with each other and with other methods in various ways in order to custom design the desired method for determining the viewing direction of the operator.

FIG. 10 is a flowchart for explaining Operation S330 of FIG. 3 in detail. In Operation S1010, the ultrasound diagnostic device 210 determines a second position to which the display 250 is to be moved in consideration of a positional relationship between the ultrasound diagnostic device 210 and an operator. Since an area in which the display 250 connected to the ultrasound diagnostic device 210 may move is limited, the second position to which the display 250 is to be moved is determined within the area where the display 250 may move.

In Operation S1020, the ultrasound diagnostic device 210 determines a movement path from the first position where the display 250 is located to the second position. In detail, the ultrasound diagnostic device 210 may determine a movement path to guide the display 250 not to collide against a person or an object while moving from the first position to the second position.

In Operation S1030, the ultrasound diagnostic device 210 changes the position of the display 250 according to the determined movement path.

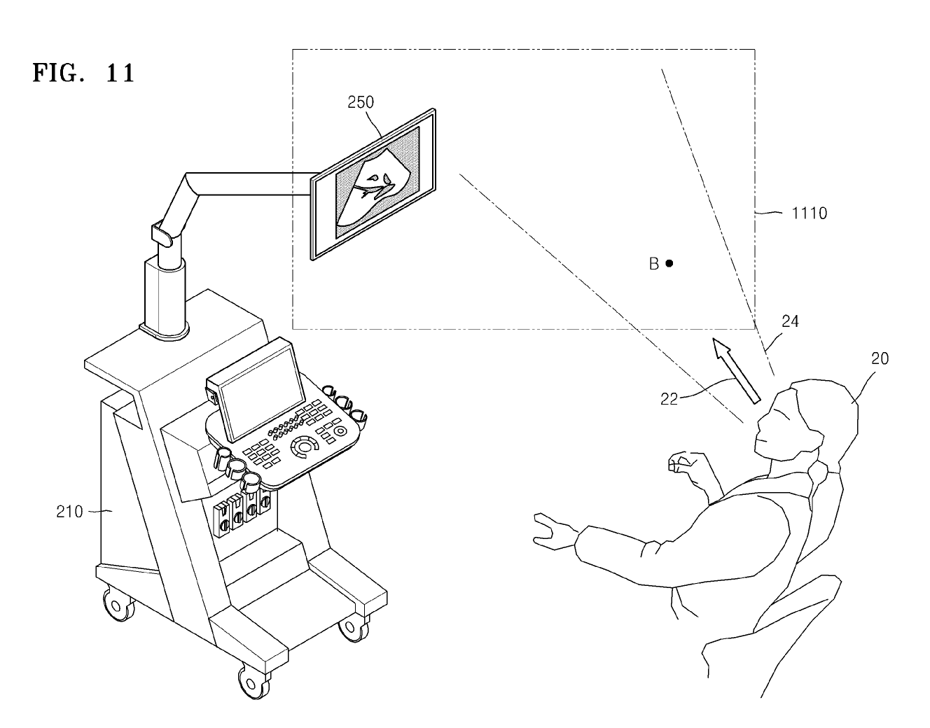

FIG. 11 illustrates a method of determining the second position considering a positional relationship between the ultrasound diagnostic device ultrasound diagnostic device 210 and the operator 20. First, the ultrasound diagnostic device 210 may determine the visible range 24 of the operator 20 based on the position and the viewing direction 22 of the operator 20. The visible range 24 of the operator 20 does not simply signify a direction in which the eyes of the operator 20 face but signifies a range in which a field of vision of the operator 20 is secured with respect to the direction in which the eyes of the operator 20 face. The visible range 24 may be determined by using statistical information about a viewing angle of an ordinary person or in various ways by the operator 20. Thus, after determining the viewing range of the operator, the ultrasound diagnostic device 210 may determine whether the operator looks at the display or whether the operator looks at other objects in the room such as the probe, the investigated person or object, or in other directions.

As illustrated in FIG. 11, a position B that is located in the visible range 24 of the operator 20 in an area 1110 within which the display 250 of the ultrasound diagnostic device 210 may move may be determined to be the second position. When there is no position that is located in the visible range 24 of the operator 20 in the area 1110 within which the display 250 may move, in other words, there is no position commonly included in the visible range 24 of the operator 20 and the area 1110 within which the display 250 may move, a position located within a predetermined distance from the visible range 24 of the operator 20 in the area 1110 within which the display 250 may move may be determined to be the second position. Thus, the display may be kept still when the operator does not look at the display.

FIG. 12 illustrates a method of determining a movement path 1210 between the first position A and the second position B from an image of an operator. FIG. 12 illustrates a depth image of an operator.

When the first position A and the second position B are determined, the ultrasound diagnostic device 210 according to the present embodiment, referring to a depth image, may determined the movement path 1210 that guides the display 250 not to collide against other persons or objects while moving from the first position A to the second position B.

Since the depth image includes information about distances of the objects or persons included in the depth image, the ultrasound diagnostic device 210 prevents the display 250 from colliding against the objects or persons considering the height and width of the display 250.

The ultrasound diagnostic device 210 may change the movement path 1210 of the display 250 when an obstacle is determined to exist on the movement path 1210 of the display 250 while the display 250 is actually moved along the movement path 1210. This is because the position of an object or a person included in the depth image may be changed during the movement of the display 250.

Weather an obstacle exists on the movement path 1210 of the display 250 may be determined from the depth image or by a distance detection sensor attached on the display 250.

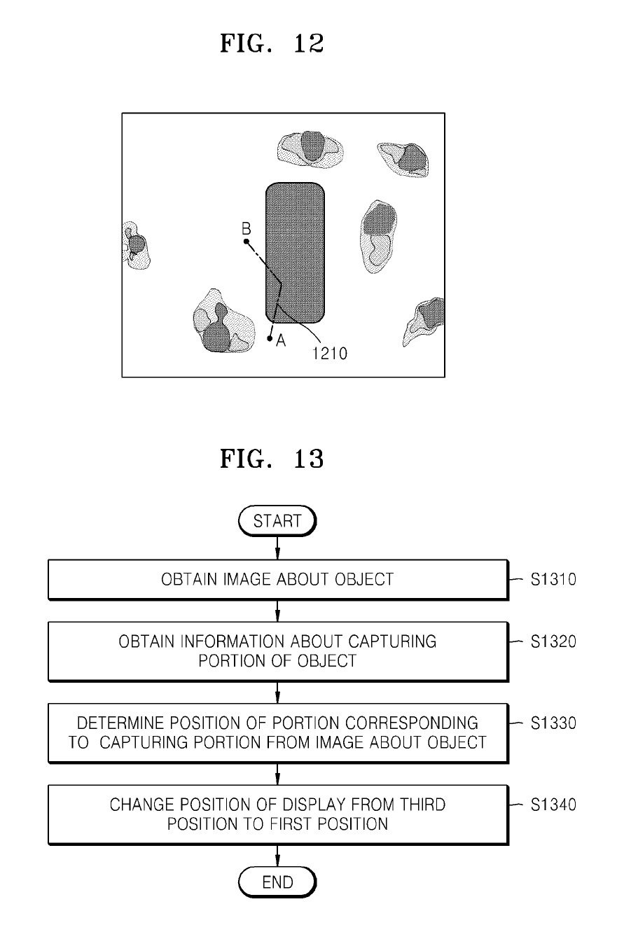

FIG. 13 is a flowchart for explaining a method of moving the display 250 of the ultrasound diagnostic device 210, according to another exemplary embodiment. The method of moving the display 250 of the ultrasound diagnostic device 210 according to FIG. 13 relates to a method of moving the display 250 in advance by using information about a portion of an object before the movement of the display 250 according to the position and the viewing direction of an operator.

In Operation S1310, the ultrasound diagnostic device 210 obtains an image of an object. The ultrasound diagnostic device 210 may receive the image of an object from an external device or server or by using a color camera, a black and white camera, or a depth camera. The image of an object may include a depth image of the object.

In Operation S1320, the ultrasound diagnostic device 210 obtains information about the portion of the object. For example, the ultrasound diagnostic device 210 may obtain information about which part of the object may be captured. The portion may include a head, a neck, an abdomen, a foot, etc. The ultrasound diagnostic device 210 may obtain the information about the portion of an object from an external server through a wired and/or wireless network.

In Operation S1330, the ultrasound diagnostic device 210 determines the position of a portion corresponding to the portion of an object, referring to an image of the object. For example, when the portion of an object is an abdomen, the ultrasound diagnostic device 210 identifies an abdomen portion of the object by applying an image processing method such as pattern matching to an image of the object and determines the position of an identified abdomen portion.

In Operation S1340, the ultrasound diagnostic device 210 changes the position of the display 250 from the third position to the first position.

According to the method of moving the display 250 of the ultrasound diagnostic device 210 according to the present embodiment, the display 250 may be moved in advance based on information about a portion of an object before a viewing direction of an operator is determined. Accordingly, when the display 250 is moved again according to the viewing direction of the operator, a movement time of the display 250 may be reduced.

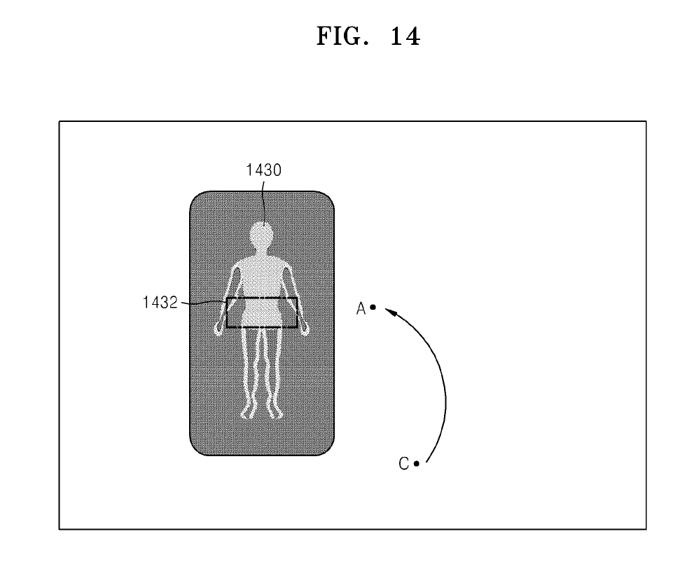

FIG. 14 illustrates a method of moving the display 250 from the third position C to the first position A. FIG. 14 illustrates a depth image of an object 1430.

When a portion of the object 1430 is an abdomen, the ultrasound diagnostic device 210 identifies an abdomen portion 1432 of the object 1430 in a depth image. Next, the ultrasound diagnostic device 210 moves the display 250 located at the third position C to the first position A close to the abdomen portion 1432 of the object.

The first position a may be determined considering an average basic posture of an operator capturing the abdomen portion 1432 of the object. For example, an average basic posture of an operator may be determined from the postures of operators capturing the abdomen portion 1432 of the object and then the first position A may be determined according to the position and viewing direction of the operator when the operator takes the average basic posture.

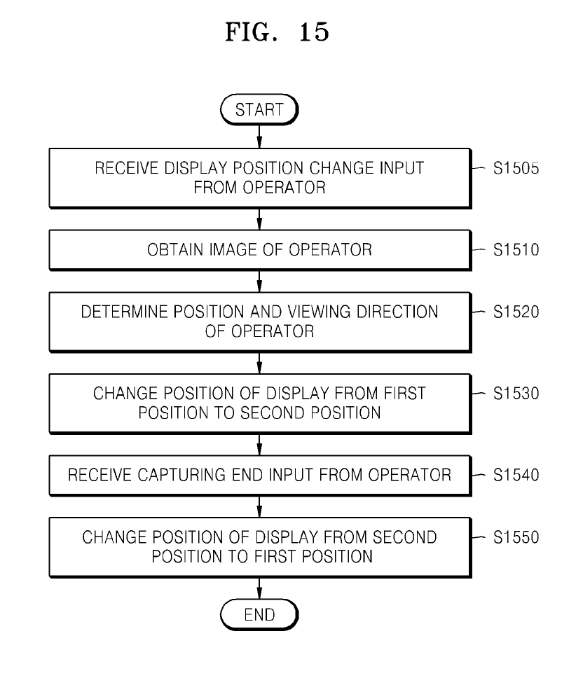

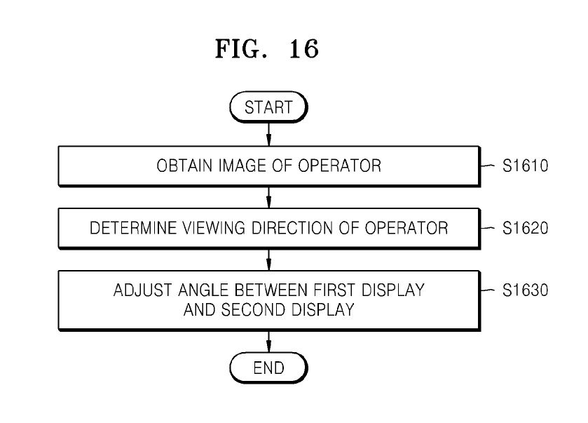

FIG. 15 is a flowchart for explaining a method of moving the display 250 of the ultrasound diagnostic device 210, according to another exemplary embodiment. Referring to FIG. 15, in Operation S1505, the ultrasound diagnostic device 210 receives an operator's input of a change of the position of the display 250. The operator may input a position change input to the ultrasound diagnostic device 210 by using at least one of a foot switch, voice, and gesture.

When the ultrasound diagnostic device 210 is located in an operation room, the operator may directly change the position of the display 250, or input a position change input, by using a hand, an object may be infected by germs because the operator's hand may be infected by the germs. Accordingly, when the operator inputs a position change by using at least one of a foot switch, voice, and gesture, the operator may be prevented from being infected by germs. The position change of the display 250 may be input by using a touch screen, a track ball, a button, etc.

In Operation S1510, the ultrasound diagnostic device 210 obtains an image of the operator. In Operation S1520, the ultrasound diagnostic device 210 determines the position and viewing direction of the operator by using an image of the operator. In Operation S1530, the ultrasound diagnostic device 210 changes the position of the display 250 from a first position to a second position according to the position and viewing direction of the operator.

In Operation 1540, the ultrasound diagnostic device 210 receives a capturing end input command from the operator. The capturing end input command may signal to the ultrasound diagnostic device that the recording or capturing of ultrasound diagnostic image data has been completed. Like the position change input, the capturing end input may include at least one of an input through a foot switch, an input through operator's voice, and an input through operator's gesture.

In Operation S1550, the ultrasound diagnostic device 210 changes the position of the display 250 from the second position to the first position according to the image capturing end input. This is to change the position of the display 250 to the original position after the image capturing ends. The ultrasound diagnostic device 210 may determine a movement path to move the display 250 from the second position to the first position from an image of the operator. When an obstacle is determined to exist on the movement path during the movement of the display 250, the ultrasound diagnostic device 210 may change the movement path of the display 250.

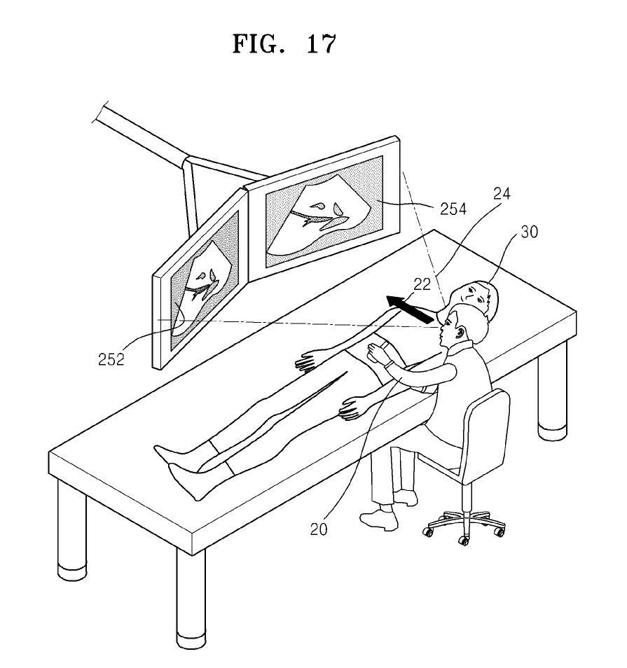

FIG. 16 is a flowchart for explaining a method of moving a display of the ultrasound diagnostic device 210, according to another exemplary embodiment. In FIG. 16, the ultrasound diagnostic device 210 may include a first display and a second display connected to each other.

In Operation S1610, the ultrasound diagnostic device 210 obtains an image of an operator. In Operation S1620, the ultrasound diagnostic device 210 determines a viewing direction of the operator from an image of the operator. Since a method of determining a viewing direction of the operator by using an image of the operator is already described above, a detailed description thereof will be omitted herein.

In Operation 1630, the ultrasound diagnostic device 210 adjusts an angle between the first and second displays according to a viewing direction of the operator. The angle between the first and second displays may be variously set by the operator.

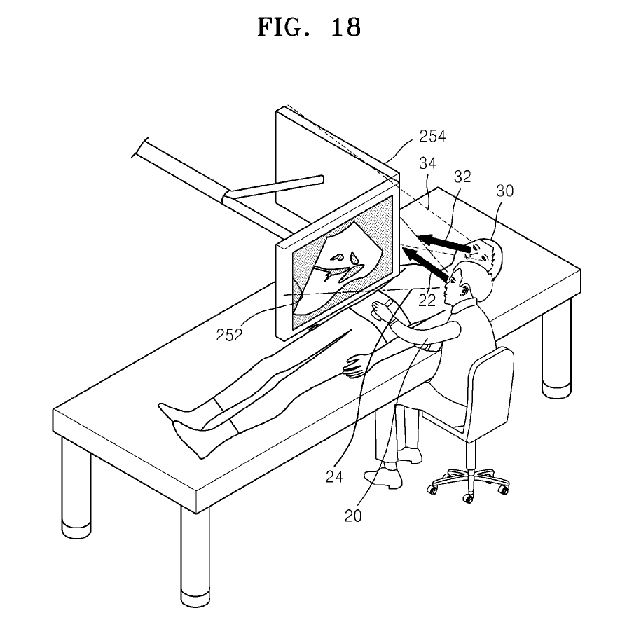

FIG. 17 illustrates a method of adjusting an angle between a first display 252 and a second display 254. Referring to FIG. 17, the ultrasound diagnostic device 210 may adjust the angle between the first display 252 and the second display 254 according to the viewing direction 22 of the operator 20. Accordingly, a screen of the first display 252 and a screen of the second display 254 may be located in the visible range 24 of the operator 20. Although FIG. 17 illustrates two displays 252 and 254 only, a person of ordinary skill in the art would understand that more than two displays may be included in the ultrasound diagnostic device.

FIG. 18 illustrates another method of adjusting an angle between the first display 252 and the second display 254. Referring to FIG. 18, the ultrasound diagnostic device 210 may determine the viewing direction 22 of the operator 20 from an image of the operator 20 and a viewing direction 32 of the object 30 from an image of the object 30. The object 30 may be a person. The ultrasound diagnostic device 210 may adjust an angle between the first display 252 and the second display 254 according to the viewing direction 22 of the user 20 and the viewing direction 32 of the object 30. Accordingly, the screen of the first display 252 may be located within the viewing range 24 of the operator 20 and the screen of the second display 254 may be located within the viewing range 34 of the object 30.

In the ultrasound diagnostic device 210 used to observe a fetus, the object 30 often wants to directly see a fetus. Thus, in the ultrasound diagnostic device 210 according to the present embodiment, the operator 20 may see the first display 252 and the object 30 may see the second display 254. In other words, referring to FIGS. 17 and 18, the ultrasound diagnostic device 210 according to the present embodiment may include two displays and the operator 20 may use both of the two displays or one of the two displays may be used by the object 30.

Conventionally, a display for an object and a display for an operator are separately connected to an ultrasound diagnostic device. In the ultrasound diagnostic device 210 according to the present embodiment, without distinguishing a display for an object and a display for an operator, two displays may be use for a variety of purposes by adjusting an angle between the two displays for convenience sake. Also, when there is a preset value by the operator 20 about the angle between two displays, the angle between the two displays may be adjusted according to the preset value.

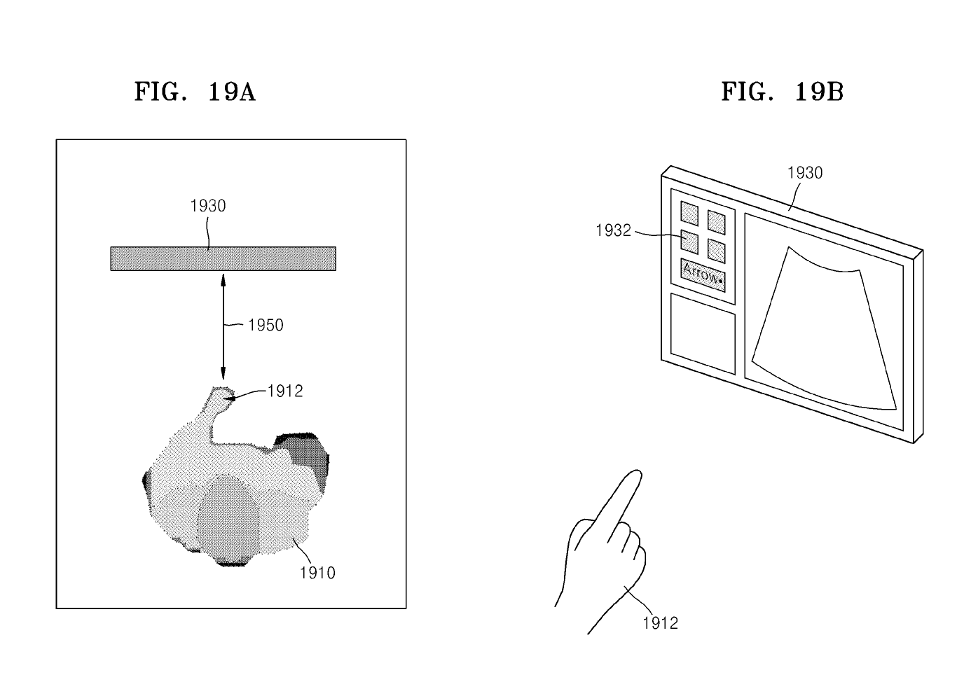

The ultrasound diagnostic device 210 according to the present embodiment may detect operator's gesture regarding a display located at the second position and perform a function of the ultrasound diagnostic device 210 corresponding to the operator's gesture. As described above, when the ultrasound diagnostic device 210 is located in an operation room, it is very important to recognize the user's gesture in order to prevent the operator's hands from being infected by germs, which will be described with reference to FIG. 19.

FIG. 19A illustrates a method of detecting gesture of an operator 1910 from a depth image. FIG. 19B illustrates a method of performing a function of the ultrasound diagnostic device 210 displayed on a display 1930 according to gesture of the operator 1910.

FIG. 19A illustrates a depth image including the user 1910 and the display 1930 located at the second position. When it is determined that a hand 1912 or a finger of the operator 1910 and the display 1930 included in the depth image are located for a predetermined time within a preset distance 1950, the ultrasound diagnostic device 210 may detect that a gesture motion by the operator 1910 is input.

The operator may have one or more identifying patches attached or painted on his hand or fingers. The patches may have specific colors, fluorescence, or shapes that the ultrasound diagnostic device may identify by using an image an color recognition software. The patches may be used to identify specific fingers or parts of the hand of the operator. Different colors and fluorescent degrees may be used to tag different fingers. Further, the above methods and devices may be combined with each other and with other methods in various ways in order to custom design the desired method for determining the gestures of the operator.

Referring to FIG. 19B, when detecting that a gesture motion is input by the operator 1910, the ultrasound diagnostic device 210 may perform a function 1932 indicated by the hand 1912 of the operator 1910 among functions of the ultrasound diagnostic device 210 displayed on the display 1930.

Also, the ultrasound diagnostic device 210 may detect the gesture motion of the operator 1910 by using a gesture detection camera attached on the display 1930. Since a method of detecting a gesture motion of the operator 1910 through an image of an operator captured by a gesture detection camera is a well-known technology to one of ordinary person in the art, a detailed description thereof will be omitted herein.

The ultrasound diagnostic device 210 may detect gesture of eyes of the operator 1910 by using an eye tracking sensor attached on the display 1930 in addition to the depth image. In detail, the eye tracking sensor may track a direction indicated by an eyeball of the operator 1910 by detecting a movement of the eyeball of the operator 1910. Then, the eye tracking sensor may identify an eye blinking gesture input, a voice input, and a foot input of the operator 1910 and perform a function corresponding to the direction indicated by an eyeball of the operator 1910 among the function displayed on the display 1930.

The ultrasound diagnostic device 210 may manually change the position of the display 1930 according to the gesture motion of the operator 1910. The operator 1910 may finely adjust the position of the display 1930 located at the second position through the gesture motion.

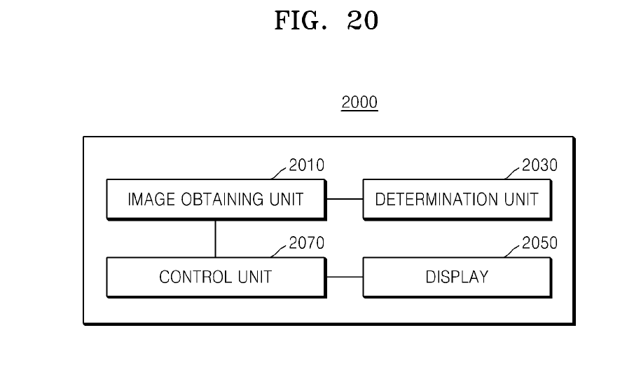

FIG. 20 is a block diagram illustrating the structure of an ultrasound diagnostic device 2000 according to an exemplary embodiment. Referring to FIG. 20, the ultrasound diagnostic device 2000 according to the present embodiment may include an image obtaining unit 2010, a determination unit 2030, a display 2050, and a control unit 2070. The image obtaining unit 2010, the determination unit 2030, and the control unit 2070 may be embodied by a microprocessor.

The image obtaining unit 2010 may receive an image of an operator or an object from an external device or obtain the image by using a color camera, a black and white camera, or a depth camera. The image of an operator or an object may include a depth image and may be an image separately captured for each of an operator and an object or an image including both of the operator and the object.

The determination unit 2030 may determine the position and viewing direction of an operator based on the image of the operator. Also, the determination unit 2030 may determine a visible range of an operator from the position and viewing direction of the operator. Since a method of determining a visible range of an operator from the image of the operator is already described above, a detailed description thereof will be omitted herein.

The display 2050 displays at least one of information about an object and an ultrasound image of the object on a screen. The information about an object may include name, age, portion, etc. of the object. The ultrasound image of an object may include a B-mode image, an M-mode image, a Doppler image, etc.

The display 2050 may include a CRT, an LCD, a PDP, an OLED, an FED, an LED, a VFD, a DLP, a PFD, a 3D display, a transparent display, etc. and may include a variety of display within a range that is obvious to one of ordinary skill in the art.

The control unit 2070 may change the position of the display 2050 from the first position to the second position according to the position and viewing direction of an operator. The control unit 2070 may determine the second position according to a relative positional relationship between the ultrasound diagnostic device 2000 and the operator before the position of the display 2050 is changed and determine a movement path from the first position to the second position referring to an image of an operator. Also, when an obstacle is determined to exist on a movement path of the display 2050 by using a distance detection sensor attached on the display 2050, the control unit 2070 changes the movement path of the display 2050 so that collision against the obstacle may be prevented.

The display 2050 of the ultrasound diagnostic device 2000 according to the present embodiment may include a first display and a second display that display at least one of information about an object and an ultrasound image of the object.

The control unit 2070 may adjust an angle between the first display and the second display. In detail, the control unit 2070 may adjust the angle between the first display and the second display according to the viewing direction of the operator such that a screen of the first display and a screen of the second display are located within a visible range of the operator, or may adjust the angle between the first display and the second display according to the viewing direction of the operator and the viewing direction of the object such that the screen of the first display is located within a visible range of the operator and the screen of the second display is located within a visible range of the object.

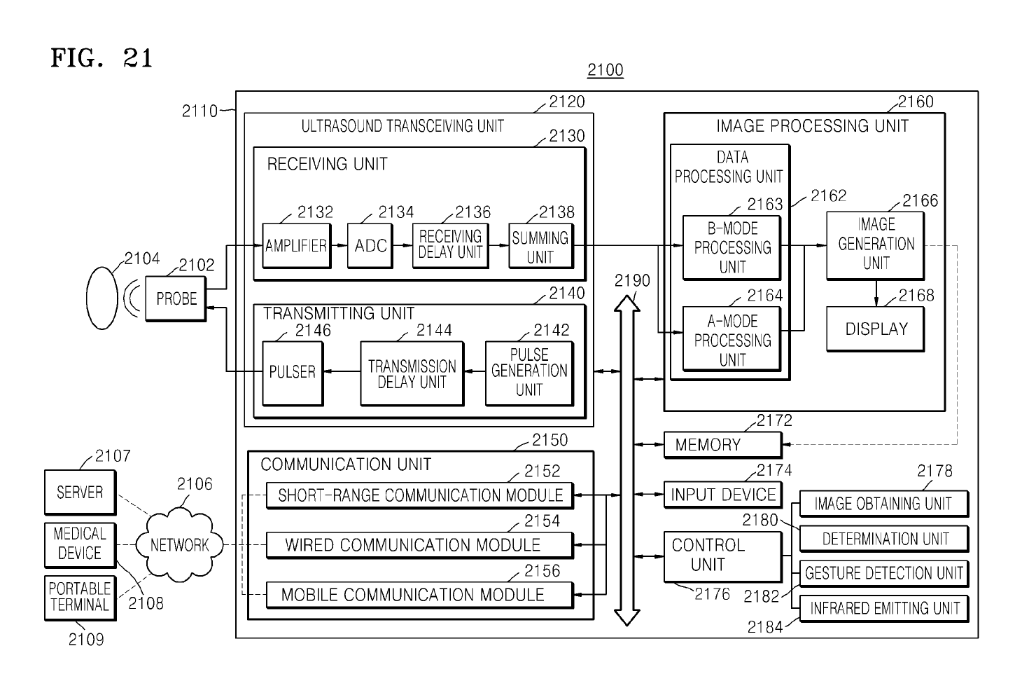

FIG. 21 is a block diagram illustrating the structure of an ultrasound diagnostic device 2100 according to another exemplary embodiment. Referring to FIG. 21, the ultrasound diagnostic device 2100 may include a probe 2102, an ultrasound transceiving unit 2120, an image processing unit 2160, a communication unit 2150, a memory 2172, an input device 2174, a control unit 2176, an image obtaining unit 2178, a determination unit 2180, a gesture detection unit 2182, and an infrared emitting unit 2184. The above-described elements may be connected to each other via a bus 2190.

The ultrasound diagnostic device 2100 may be embodied in not only a cart type or a portable type. A portably ultrasound diagnostic device may be, for example, a PACS viewer, a smartphone, a laptop computer, a PDA, a tablet PC, etc., but the exemplary embodiments are not limited thereto.

The probe 2102 transmits an ultrasound signal to an object 2104 according to a driving signal applied by the ultrasound transceiving unit 2120 and receives a response signal reflected from the object 2104. The probe 2102 includes a plurality of transducers. Each transducer generates an ultrasound wave that is acoustic energy and vibrates according to a transferred electrical signal. Also, the probe 2102 may be connected with a main body of the ultrasound diagnostic device 2100 in a wired or wireless way. The ultrasound diagnostic device 2100 may be provided with a plurality of the probes 2102 according to an embodiment type.

The transmission unit 2140 supplies a driving signal to the probe 2102 and includes a pulse generation unit 2142, a transmission delay unit 2144, and a pulser 2146. The pulse generation unit 2142 generates pulses for forming a transmission ultrasound wave according to a predetermined pulse repetition frequency (PRF). The transmission delay unit 2144 applies a delay time to determine transmission directionality to a pulse. Each pulse to which a delay time is applied corresponds to each of a plurality of piezoelectric vibrators included in the probe 2102. The pulse 2146 applies a driving signal or a driving pulse to the probe 2102 at a timing corresponding to each pulse to which the delay time is applied.

The receiving unit 2130 may generate ultrasound data by processing a response signal received from the probe 2102 and may include an amplifier 2132, an analog-to-digital converter (ADC) 2134, a receiving delay unit 2136, and a summing unit 2138. The amplifier 2132 amplifies a response signal for each channel. The ADC 2134 performs analog-to-digital conversion on an amplified response signal. The receiving delay unit 2136 applies a delay time to determine reception directionality to a digitally converted response signal. The summing unit 2138 generates ultrasound data by summing the response signal processed by the receiving delay unit 2136.

The image processing unit 2160 generates and displays an ultrasound image through a scan conversion process on the ultrasound data generated by the ultrasound transceiving unit 2120. An ultrasound image may be presented not only as a gray scale ultrasound image obtained by scanning an object according to an amplitude (A) mode, a brightness (B) mode, and a motion (M) mode, but also as a Doppler image representing a motion of an object. A Doppler image may include a blood flow Doppler image (referred to as a color Doppler image) indicating the flow of blood, a tissue Doppler image indicating a motion of tissue, and a spectral Doppler image displaying a movement speed of an object as a waveform.

A B-mode processing unit 2163 extracts a B-mode component from ultrasound data and processes the extracted component. An image generation unit 2166 may generate an ultrasound image, in which signal strength is presented by brightness, based on the B-mode component extracted by the B-mode processing unit 2163.

Likewise, the Doppler processing unit 2164 extracts a Doppler component from the ultrasound data and the image generation unit 2166 may generate a Doppler image in which a movement of an object is presented by a color or a waveform based on the extracted Doppler component.

The image generation unit 2166 according to the present embodiment may generate a 3D ultrasound image through a volume rendering process on volume data and generate an elastic image obtained by visualizing a degree of deformation of the object 2104 according to pressure. Furthermore, the image generation unit 2166 may express various pieces of additional information by text or graphics on the ultrasound image. The generated ultrasound image may be stored in the memory 2172.

The display 2168 displays the generated ultrasound image. The display 2168 may display not only an ultrasound image but also various pieces of information processed by the ultrasound diagnostic device 2100 on a screen through a graphic user interface (GUI). The ultrasound diagnostic device 2100 may include two more displays 2168 according to an embodiment type. Also, as described above, the display 2168 may be connected to the ultrasound diagnostic device 2100 via an arm having a degree of freedom of 6 axes and may be moved to a certain position by the arm. However, aspects of the exemplary embodiments are not limited thereto, such that other systems for moving the display, which may have other number of degrees of freedom, may be used.

The communication unit 2150 is connected to a network 2106 in a wired or wireless way to communicate with an external device or server. The communication unit 2150 may communicate data with a server or other medical device in a hospital via a picture archiving and communication system (PACS). Also, the communication unit 2150 may communicate data according to a digital imaging and communications in medicine (DICOM).

The communication unit 2150 may transceive data related to diagnosis of an object such as an ultrasound image, ultrasound data, Doppler data, etc. of the object through the network 2106 and also transceive a medical image captured by other medical device such as a CT, an MRI, an X-ray, etc. Furthermore, the communication unit 2150 may receive information about a diagnosis history or treatment schedule of a patient from a server and use the received information for diagnosis of the object. The communication unit 2150 may perform data communication not only with a server or medical devices in a hospital but also with a portable terminal of a medical doctor or a patient.

The communication unit 2150 may perform communication with an external device or server and obtain information about a portion of an object. The determination unit 2180 may determine the position of a portion corresponding to the portion of an object from an image of the object and change the position of the display 2168 from the third position to the first position according to the position of the determined portion. Accordingly, the 2100 may move in advance the display 2158 according to information about the portion of the object so as to reduce a movement time to move the display 2168 along the viewing direction of the operator.

The communication unit 2150 may receive a position change input of the display 2168 by using at least one of a foot switch, voice, and gesture, by the operator. In this case, the control unit 2176 may determine the viewing direction of the operator according to the operator's position change input and then change the position of the display 2168 from the first position to the second position. Also, the control unit 2176 may change the position of the display 2168 from the third position to the first position based on information about the portion of the object according to the operator's position change input. The communication unit 2150 may received a capturing end input by using at least one of a foot switch, voice, and gesture by the operator. The control unit 2176 may change the position of the display 2168 from the first position to the second or third position according to the capturing end input.

The communication unit 2150 is connected to the network 2106 in a wired or wireless way to communicate data with a server 2107, a medical device 2108, or a portable terminal 2109. The communication unit 2150 may include one or more constituent elements that enable communication with an external device, for example, a short-range communication module 2152, a wired communication module 2154, and a mobile communication module 2156.

The short-range communication module 2152 signifies a module for short-range communication within a predetermined distance. The short-range communication technology includes, for example, wireless LAN, Wi-Fi, Bluetooth, Zigbee, Wi-Fi Direct (WFD), ultra wideband (UWB), infrared data association (IrDA), Bluetooth low energy (BLE), near field communication (NFC), etc., but the exemplary embodiments are not limited thereto.

The wired communication module 2154 signifies a module for communication using an electrical signal or an optical signal. A wired communication technology according to an embodiment may include a pair cable, a coaxial case, an optical fiber cable, an Ethernet cable, etc.

The mobile communication module 2156 transceives a wireless signal with at least one of a local station, an external terminal, and a server on a mobile communication network. The wireless signal may include a variety of forms of data according to communication of a voice call signal, a video call signal, or a text and/or multimedia message.

The memory 2172 stores various pieces of information processed by the ultrasound diagnostic device 2100. For example, the memory 2172 may store medical data related to diagnosis of an object such as ultrasound data, an ultrasound image, etc. that is input or output, or an algorithm or a program executed in the ultrasound diagnostic device 2100.

The memory 2172 may be embodied by a variety of types of storage media such as flash memory, a hard disk, EEPROM, etc. Also, the ultrasound diagnostic device 2100 may employ a web storage or a cloud server performing a storage function of the memory 2172 on a web.

The input device 2174 signifies a unit for receiving an input of data from an operator to control the ultrasound diagnostic device 2100. The input device 2174 may include hardware such as a keypad, a mouse, a touch panel, a touch screen, a trackball, a jog switch, etc., but the exemplary embodiments are not limited thereto and the input device 2174 may further include various input units such as an electrocardiogram measuring module, a respiration measuring module, a voice recognition sensor, a gesture recognition sensor, a fingerprint recognition sensor, an iris recognition sensor, a depth sensor, a distance sensor, etc.

The control unit 2176 controls the overall operation of the ultrasound diagnostic device 2100. In other words, the control unit 2176 may control the operations among the probe 2102, the ultrasound transceiving unit 2120, the image processing unit 2160, the communication unit 2150, the memory 2172, the input device 2174, the image obtaining unit 2178, the determination unit 2180, the gesture detection unit 2182, and the infrared emitting unit 2184. The control unit 2176 may change the position of the display 2168 by controlling the arm to which the display 2168 is connected.

The image obtaining unit 2178 may receive an image of an operator or the object 2104 from an external device or an external server 2107 and obtain the image of an operator or the object 2104 by using a color camera, a black and white camera, or a depth camera.

The determination unit 2180 may determine the position, viewing direction, and visible range of an operator or the object 2104 based on the image of an operator or the object 2104.

The gesture detection unit 2182 may detect gestures of an operator with respect to the display 2168 located at the second position. The control unit 2176 may perform a function of the ultrasound diagnostic device 2100 corresponding to the gesture motion detected by the gesture detection unit 2182. The gesture detection unit 2182 may detect gesture of an operator by using the image of an operator, a gesture detection camera attached on the display 2168, or an eye tracking sensor attached on the display 2168.

The infrared emitting unit 2184 emits an infrared ray into a predetermined space including an operator or the ultrasound diagnostic device 2100. The control unit 2176 may determine the position of an object that reflects or absorbs the emitted infrared ray as the position of the probe 2102 and determine as the operator a person who is the closest to the position of the probe 2102 among a plurality of persons included in the image of an operator. When the image obtaining unit 2178 of the ultrasound diagnostic device 2100 according to the present embodiment includes a depth camera that uses an infrared method, the position of the probe 2102 may be determined by using an infrared ray emitted by the depth camera, instead of the infrared ray emitted by the infrared emitting unit 2184.

Part or the whole of the probe 2102, the ultrasound transceiving unit 2120, the image processing unit 2160, the communication unit 2150, the memory 2172, the input device 2174, the control unit 2176, the image obtaining unit 2178, the determination unit 2180, the gesture detection unit 2182, and the infrared emitting unit 2184 may be operated by a software module, but the exemplary embodiments are not limited thereto and part of the above-described structure may be operated by hardware. Also, at least part of the ultrasound transceiving unit 2120, the image processing unit 2160, and the communication unit 2150 may be included in the control unit 2176, but the exemplary embodiments are not limited to such an embodiment.

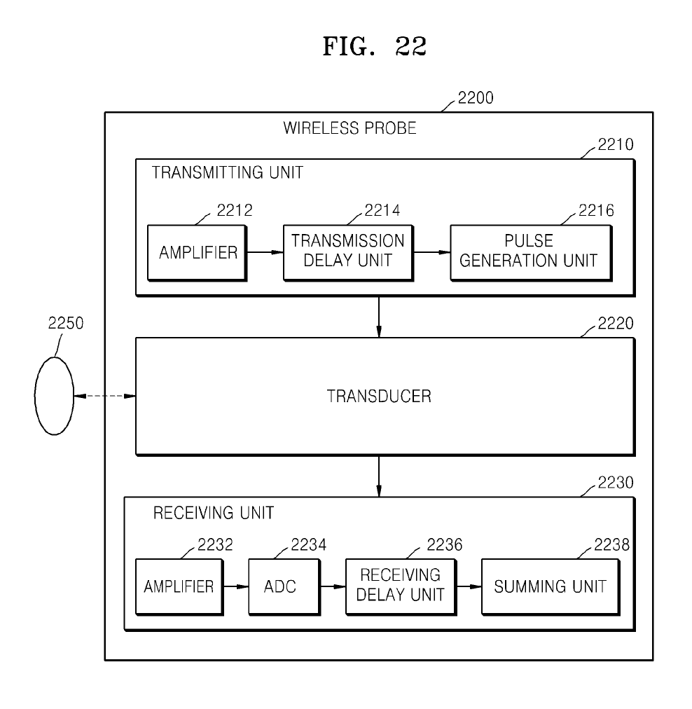

FIG. 22 is a block diagram illustrating the structure of a wireless probe 2200 that may be connected to an ultrasound diagnostic device, according to an exemplary embodiment. The wireless probe 2200 of FIG. 22 may include a plurality of transducers as described in FIG. 21 and may include part or the whole of the structure of the ultrasound transceiving unit 2120 of FIG. 21.

The wireless probe 2200 according to the present embodiment of FIG. 22 includes a transmitting unit 2210, a transducer 2220, and a receiving unit 2230, whose structures are already described with reference to FIG. 21 and thus detailed descriptions thereof will be omitted herein. The wireless probe 2200 may selectively include a receiving delay unit 2236 and a summing unit 2238 according to an embodiment type thereof.

The wireless probe 2200 may transmit an ultrasound signal to an object 2250 and receive a response signal therefrom, or generate ultrasound data and wirelessly transmit the generated ultrasound data to the ultrasound diagnostic device 2100 of FIG. 21.

The exemplary embodiments can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, etc. The computer readable recording medium can also be distributed over network coupled computer systems so that the computer readable code is stored and executed in a distributed fashion.

While a few exemplary embodiments have been shown and described, it will be understood by those skilled in the art that various changes in form and details may be made therein without departing from the spirit and scope of the inventive concept as defined by the appended claims.

In addition, many modifications can be made to adapt a particular situation or material to the teachings of the present disclosure without departing from the essential scope thereof. Therefore, it is intended that the present disclosure not be limited to the particular exemplary embodiments disclosed as the best mode contemplated for carrying out the present disclosure, but that the present disclosure will include all embodiments falling within the scope of the appended claims.

What is claimed is:

1. A method of moving a display of an ultrasound diagnostic device, the method comprising:
obtaining an image comprising an operator of the ultrasound diagnostic device, the operator performing a capturing of an ultrasound image of an object by using the ultrasound diagnostic device;
determining a position and a viewing direction of the operator based on the image comprising the operator; and
changing a position of the display connected to the ultrasound diagnostic device from a first position to a second position according to the position and the viewing direction of the operator.

2. The method of claim 1, wherein the changing of the position of the display comprises:
determining a viewing range of the operator based on the position and the viewing direction of the operator; and
changing the position of the display from the first position to the second position such that a screen of the display is located in the determined viewing range of the operator.

3. The method of claim 1, wherein the determining of the viewing direction of the operator comprises:
determining a posture of the operator based on the image of the operator; and
determining the viewing direction of the operator by using the determined posture of the operator.

4. The method of claim 3, wherein the determining of the posture of the operator comprises determining the posture of the operator based on at least one of a shape and a positional relationship of a head part and a body part of the operator included in the image of the operator.

5. The method of claim 1, wherein the image comprising the operator further comprises a support table supporting the object, and
the determining of the viewing direction of the operator comprises determining a positional relationship between the operator and the support table comprised in the image of the operator.

6. The method of claim 1, wherein the changing of the position of the display comprises determining the second position, and the determining of the second position comprises determining a positional relationship between the ultrasound diagnostic device and the operator.

7. The method of claim 2, wherein the changing of the position of the display comprises determining a position located in the viewing range of the operator and in a space within which the display is movable as the second position.

8. The method of claim 1, wherein:
the obtaining of the image of the operator comprises obtaining a depth image of the operator, and
the determining of the position and the viewing direction of the operator comprises determining the position and the viewing direction of the operator based on the depth image of the operator.

9. The method of claim 8, wherein the obtaining of the depth image comprises obtaining a depth image of the operator by using a depth camera.

10. The method of claim 8, wherein the obtaining of the depth image comprises:
obtaining images of the operator by using at least two color cameras; and
obtaining a depth image of the operator by applying stereo matching to the images of the operator obtained by using the at least two color cameras.

11. The method of claim 1, wherein the changing of the position of the display comprises determining a movement path from the first position to the second position based on information about a position of at least one of a person and an object comprised in the image comprising the operator.

12. The method of claim 11, wherein the determining of the movement path comprises determining the movement path from the first position to the second position such that the display does not collide with at least one of the person and the object included in the image comprising the operator.

13. The method of claim 1, wherein the changing of the position of the display comprises changing from a first movement path to a second movement path of the display upon determining that an obstacle exists on the first movement path of the display;
wherein no obstacle exists on the second movement path; and
wherein the first and second movement paths are moving paths between the first position of the display and the second position of the display.

14. The method of claim 1, further comprising:
obtaining information about a portion of the object, the portion being a portion to which ultrasound is applied;
obtaining an image of the object;
determining a position of a portion corresponding to the portion in the image of the object; and
changing a position of the display from a third position to the first position according to the determined position of the portion.

15. The method of claim 14, wherein the obtaining of the image of the object comprises obtaining a depth image of the object.

16. The method of claim 1, wherein the determining of the position and the viewing direction of the operator comprises determining a person who is the closest to a position of a probe of the ultrasound diagnostic device when a plurality of persons are comprised in the image comprising the operator.

17. The method of claim 16, wherein the determining of the person who is the closest to the position of the probe of the ultrasound diagnostic device as the operator comprises:
determining the position of the probe by using infrared or short-range communication; and
determining a person who is the closest to the position of the probe, based on the position of the probe and the image of the operator, as the operator.

18. The method of claim 1, wherein the display comprises a first display and a second display, and
the method further comprising adjusting an angle between the first display and the second display.

19. The method of claim 18, wherein the adjusting of the angle between the first display and the second display comprises adjusting the angle between the first display and the second display such that a screen of the first display and a screen of the second display are located at the viewing direction of the operator.

20. The method of claim 18, wherein the adjusting of the angle between the first display and the second display comprises adjusting the angle between the first display and the second display so that a screen of the first display is located at the viewing direction of the operator and the screen of the second display is located at a viewing direction of the object.

21. The method of claim 1, further comprising adjusting at least one of a brightness value, a contrast value, and a tilting angle of the display located at the second position according to a preset value.

22. The method of claim 1, further comprising:
detecting a gesture of the operator with respect to the display located at the second position; and
performing a function of the ultrasound diagnostic device corresponding to the gesture of the operator.

23. The method of claim 22, wherein the detecting of the gesture of the operator comprises detecting a gesture of the operator by using the image of the operator.

24. The method of claim 23, wherein the detecting of the gesture of the operator comprises performing a function corresponding to a position indicated by a hand of the operator among functions displayed on the display when it is determined, based on the image of the operator, that the hand of the operator and the display are located within a preset distance for a preset time.

25. The method of claim 22, wherein the detecting of the gesture of the operator comprises detecting a gesture of the operator by using a gesture detection camera attached to the display.

26. The method of claim 22, wherein the detecting of the gesture of the operator comprises:
detecting a gesture or movement of an eye of the operator by using an eye tracking sensor attached to the display or attached to a headset of the operator; and
performing a function of the ultrasound diagnostic device corresponding to the detected gesture or movement of the eye of the operator.

27. The method of claim 1, further comprising receiving a display position change input from the operator.

28. The method of claim 27, wherein the display position change input comprises at least one of an input through a foot switch connected to the ultrasound diagnostic device, an input through operator's voice, and an input through operator's gesture.

29. The method of claim 1, further comprising:
receiving a capturing end input by the operator or receiving input from the operator to end image capturing; and
changing the position of the display from the second position to the first position according to the capturing end input.

30. A method of moving a display of an ultrasound diagnostic device, the method comprising:
obtaining an image of an object whose ultrasound image is to be captured by using the ultrasound diagnostic device;
obtaining information about a portion of the object;
determining a position of a first portion corresponding to the portion based on the image of the object; and
changing a position of the display connected to the ultrasound diagnostic device from a first position to a second position according to the determined position of the first portion.

31. A method of moving a display of an ultrasound diagnostic device, the method comprising:
obtaining an image of an operator performing ultrasound imaging of an object by using the ultrasound diagnostic device;
determining a viewing direction of the operator from the obtained image of the operator; and
adjusting an angle between a first display and a second display that are connected to the ultrasound diagnostic device, according to the viewing direction of the operator.

32. The method of claim 31, wherein the adjusting of the angle between the first display and the second display comprises adjusting the angle between the first display and the second display such that a screen of the first display and a screen of the second display are located at the viewing direction of the operator.

33. The method of claim 31, further comprising:
obtaining an image of the object; and
determining a viewing direction of the object based on the image of the object,
wherein the adjusting of the angle between the first display and the second display comprises adjusting the angle between the first display and the second display such that a screen of the first display is located at the viewing direction of the operator and the screen of the second display is located at a viewing direction of the object.

34. A non-transitory computer readable storage medium having recorded thereon a program for executing a method comprising:
obtaining an image comprising an operator of an ultrasound diagnostic device, the operator performing the capturing of an ultrasound image of an object by using the ultrasound diagnostic device;
determining a position and a viewing direction of the operator based on the image comprising the operator; and
changing a position of a display connected to the ultrasound diagnostic device from a first position to a second position according to the position and the viewing direction of the operator.

35. An ultrasound diagnostic device comprising:
an image obtaining unit for obtaining an image comprising an operator of the ultrasound diagnostic device, the operator performing the capturing of an ultrasound image of an object;
a determination unit for determining a position and a viewing direction of the operator based on the image comprising the operator;
a display for displaying at least one of an information of the object and an ultrasound image of the object; and
a control unit for changing a position of the display from a first position to a second position according to the position and the viewing direction of the operator.

36. The ultrasound diagnostic device of claim 35, wherein the determination unit determines a viewing range of the operator based on the position and the viewing direction of the operator, and
the control unit changes the position of the display from the first position to the second position so that a screen of the display is located within the viewing range of the operator.

37. The ultrasound diagnostic device of claim 35, wherein the determination unit determines a posture of the operator based on the image comprising the operator and determines the viewing direction of the operator by using the determined posture of the operator.

38. The ultrasound diagnostic device of claim 37, wherein the determination unit determines the posture of the operator based on at least one of a shape and a positional relationship of a head part and a body part of the operator comprised in the image of the operator.

39. The ultrasound diagnostic device of claim 35, wherein the image of the operator comprises a support table supporting the object, and
the determination unit determines the viewing direction of the operator based on a positional relationship between the operator and the support table comprised in the image comprising the operator.

40. The ultrasound diagnostic device of claim 35, wherein the control unit determines the second position based on a positional relationship between the ultrasound diagnostic device and the operator.

41. The ultrasound diagnostic device of claim 36, wherein the control unit determines a position located in the viewing range of the operator and in a space within which the display is movable as the second position.

42. The ultrasound diagnostic device of claim 35, wherein the image obtaining unit obtains a depth image of the operator, and
the determination unit determines the position and the viewing direction of the operator based on the depth image of the operator.

43. The ultrasound diagnostic device of claim 42, wherein the image obtaining unit comprises a depth camera for obtaining a depth image of the operator.

44. The ultrasound diagnostic device of claim 42, wherein the image obtaining unit comprises at least two color cameras for obtaining images of the operator and obtains a depth image of the operator by applying stereo matching to the images of the operator obtained by using the at least two color cameras.

45. The ultrasound diagnostic device of claim 35, wherein the control unit determines a movement path from the first position to the second position based on information about a position of at least one of a person and an object comprised in the image comprising the operator.

46. The ultrasound diagnostic device of claim 45, wherein the control unit determines the movement path from the first position to the second position such that the display does not collide against at least one of the person and the object included in the image of the operator.

47. The ultrasound diagnostic device of claim 35, wherein the display comprises a distance detection sensor that detects an obstacle located within a predetermined distance.

48. The ultrasound diagnostic device of claim 47, wherein the control unit changes the movement path of the display from a first movement path to a second movement path when an obstacle detected by the distance detection sensor exists on the first movement path of the display when the position of the display is changed from the first position to the second position along the first path; and
wherein no obstacle exists on the second movement path; and
wherein the first and second movement paths are moving paths between the first position of the display and the second position of the display.

49. The ultrasound diagnostic device of claim 35, further comprising a communication unit obtaining information about a portion of the object, the portion being a portion to which ultrasound is applied;
wherein the image obtaining unit obtains an image of the object, the determination unit determines a position of a portion corresponding to the portion in the image of the object, and the control unit changes a position of the display from a third position to the first position according to a determined position of the portion.

50. The ultrasound diagnostic device of claim 35, wherein the determination unit determines a person who is the closest to a position of a probe of the ultrasound diagnostic device when a plurality of persons are comprised in the image comprising the operator.

51. The ultrasound diagnostic device of claim 50, further comprising an infrared emitting unit that emits an infrared ray,
wherein the determination unit determines a position of an object that reflects or absorbs the infrared ray emitted by the infrared emitting unit, as a position of the probe.

52. The ultrasound diagnostic device of claim 35, wherein the display comprises a first display and a second display, and the control unit adjusts an angle between the first display and the second display.

53. The ultrasound diagnostic device of claim 52, wherein the control unit adjusts the angle between the first display and the second display such that a screen of the first display and a screen of the second display are located at the viewing direction of the operator.

54. The ultrasound diagnostic device of claim 52, wherein the control unit adjusts the angle between the first display and the second display such that a screen of the first display is located at the viewing direction of the operator and the screen of the second display is located at a viewing direction of the object.

55. The ultrasound diagnostic device of claim 35, wherein the control unit adjusts at least one of a brightness value, a contrast value, and a tilting angle of the display located at the second position according to a preset value.

56. The ultrasound diagnostic device of claim 35, further comprising a gesture detection unit that detects a gesture of the operator with respect to the display located at the second position,
wherein the control unit performs a function of the ultrasound diagnostic device corresponding to the gesture of the operator.

57. The ultrasound diagnostic device of claim 56, wherein the gesture detection unit detects a gesture of the operator by using the image comprising the operator.

58. The ultrasound diagnostic device of claim 57, wherein the gesture detection unit determines, based on the image of the operator, whether a hand of the operator and the display are located within a preset distance for a preset time and
the control unit performs a function corresponding to a position indicated by the hand of the operator among functions displayed on the display.

59. The ultrasound diagnostic device of claim 56, wherein the gesture detection unit comprises a gesture detection camera that is attached on the display and detects a gesture of the operator.

60. The ultrasound diagnostic device of claim 56, wherein the gesture detection unit comprises an eye tracking sensor that is attached to the display and detects a gesture or a movement of an eye of the operator; and
the control unit performs a function of the ultrasound diagnostic device corresponding to the detected gesture or movement of the eye of the operator.

61. The ultrasound diagnostic device of claim 35, further comprising a communication unit that receives a display position change input from the operator.

62. The ultrasound diagnostic device of claim 61, wherein the display position change input comprises at least one of an input through a foot switch connected to the ultrasound diagnostic device, an input through operator's voice, and an input through operator's gesture.

63. The ultrasound diagnostic device of claim 35, further comprising a communication unit that receives a capturing end input by the operator or an input from the operator to end image capturing,
wherein the control unit changes the position of the display from the second position to the first position according to the capturing end input.

64. An ultrasound diagnostic device comprising:
an image obtaining unit for obtaining an image of an object whose ultrasound image is to be captured;
a communication unit for obtaining information about a portion of the object, the portion being a portion to which ultrasound is applied;
a determination unit for determining a position of a portion corresponding to the portion based on the image of the object;
a display for displaying at least one of an information of the object and an ultrasound image of the object; and
a control unit for changing a position of the display from a first position to a second position according to the determined position of the portion.

65. An ultrasound diagnostic device comprising:
an image obtaining unit for obtaining an image of an operator who captures an ultrasound image of an object;
a determination unit for determining a viewing direction of the operator from the image of the operator;
a first display and a second display for displaying at least one of an information of the object and the ultrasound image of the object; and
a control unit for adjusting an angle between the first display and the second display that are connected to the ultrasound diagnostic device, according to the viewing direction of the operator.

66. The ultrasound diagnostic device of claim 65, wherein the control unit adjusts the angle between the first display and the second display such that a screen of the first display and a screen of the second display are located at the viewing direction of the operator.

67. The ultrasound diagnostic device of claim 65, wherein the image obtaining unit obtains an image of the object,
the determination unit determines a viewing direction of the object based on the image of the object, and
the control unit adjusts the angle between the first display and the second display such that a screen of the first display is located at the viewing direction of the operator and the screen of the second display is located at a viewing direction of the object.

68. The ultrasound diagnostic device of claim 65, wherein the image obtaining unit obtains a depth image of the operator, and the determination unit determines the viewing direction of the operator from the depth image of the operator.
